(19) United States
(12) Patent Application Publication
(10) Pub. No.: US 2024/0216339 A1
(43) Pub. Date: Jul. 4, 2024

(54) METHODS OF TREATING NEUROPSYCHIATRIC DISORDERS

(71) Applicant: GILGAMESH PHARMACEUTICALS, INC., New York, NY (US)

(72) Inventor: Jonathan SPORN, New York, NY (US)

(73) Assignee: GILGAMESH PHARMACEUTICALS, INC., New York, NY (US)

(21) Appl. No.: 18/294,360

(22) PCT Filed: Jun. 8, 2022
(86) PCT No.: PCT/US2022/032748

§ 371 Date: Feb. 1, 2024

Related U.S. Application Data
(60) Provisional application No. 63/208,339, filed on Jun. 8, 2021.
(60) Provisional application No. 63/298,493, filed on Jan. 11, 2022.

(51) Int. Cl. A61K 31/4192; A61K 31/4045; A61K 31/675; A61P 25/00

(52) U.S. Cl. CPC A61K 31/4192; A61K 31/4045; A61K 31/675; A61P 25/00

(57) ABSTRACT

The disclosure provides, in part, a method of treating a neuropsychiatric disorder in a patient in need thereof, comprising administering to the patient an effective amount of an orexin receptor antagonist and further comprising administering to the patient an effective amount of a serotonin 2A receptor agonist or an NMDA receptor antagonist.

### BACKGROUND

The orexin system is involved in regulation of sleep and in limbic related emotional processing and in regulating stress response. Dual orexin receptor antagonists are approved for insomnia and insomnia are important symptoms in depression and may cause worsening of depression and anxiety. There remains a need to identify treatments comprising an antagonist therapy combined with psychedelic-related drugs useful in a variety of neuropsychiatric conditions.

### SUMMARY

The present disclosure provides, among other things, a method of treating a neuropsychiatric disorder comprising administrating to a patient in need thereof a pharmaceutically effective amount of a combination comprising an orexin receptor antagonist and a serotonin receptor agonist or an NMDA receptor antagonist.

In some embodiments, the serotonin receptor agonist is selected from those compounds that activate the 5-HT2a receptor and induce psychedelic effects.

In some embodiments, a method of treating a neuropsychiatric disorder includes one or more behavioral approaches. In some embodiments, a behavioral approach includes, but is not limited to dream incubation, pairing critical learning periods the day prior to administration of a combination.

In some embodiments, the present disclosure includes treatment of a variety of psychiatric illnesses as well as a means for problem solving in healthy individuals. Without being tied to any particular theory, the methods described herein have a low risk of abuse or misuse because methods described herein generally lead to sleep. In contrast to recreational use, taking a larger amount of a combination disclosed here, may be either dysphoric or less effective (i.e., lead to deeper sleep than is ideal).

In some embodiments, rapid eye movement sleep (REM) may be more effective in working through emotional connections in dreams since orexin receptor antagonists may increase dream sleep and psychedelic drugs may enhance visual imagery and neuroplasticity that occurs during sleep. Anecdotally, individuals have reported that suvorexant allowed them to sleep while using low doses of the psychedelic psilocybin and that the combination resulted in dreams with dramatic detail, positive emotional tone, and next day enhanced mood and/or self-rated enhancement of cognition. Further, individuals who fell asleep while taking LSD have anecdotally reported next day enhanced creativity and vigor more than 12 hours after the dose despite initially forgetting they took the LSD.

In some embodiments, cognitive or emotional learning is provided within a short time prior to sleep induction. This will allow the combination of the serotonin agonist and the orexin receptor antagonist to facilitate memory and learning consolidation via active consolidation systems that transfers engrams that are labile into stable long-term memories to promote behavioral change. During slow-wave sleep (SWS) there is reactivation of encoded information in the hippocampus and replay of ensemble firing patterns in a strict temporal order with compression for system consolidation. This stabilization of new learning will be important for improvement in an array of neuropsychiatric illnesses. The sleep induction can be at night or at another time of day following the learning or training affectively/cognitively, such as that resulting from various forms of psychotherapy administered by a therapist or via a digital application.

Excessive arousal has a role in the pathophysiology of major depressive disorder (MDD). Seltorexant (JNJ-42847922/MIN-202) is a selective antagonist of the human orexin-2 receptor (OX2R) that may normalize excessive arousal and thereby attenuate depressive symptoms. In a small study, ten days of treatment with seltorexant (and not diphenhydramine) resulted in a significant improvement of core depressive symptoms compared to placebo; the antidepressant efficacy of seltorexant was maintained with continued treatment up to 28 days and was seen at day 11. Compared to placebo, the antidepressant efficacy of seltorexant coincided with an overall increase in (left posterior) EEG power and a relative increase in delta power and decrease in theta, alpha, and beta power during stage 2 sleep. In MDD, a 24-hour CSF sampling study found elevated orexin release in depressed versus control subjects along with blunted orexin levels across the circadian period (Salomon 2003). In contrast, the dual OX1/2 (DORA) antagonist suvorexant is an approved treatment for insomnia but not for MDD. Further, the DORA filorexant did not separate from placebo in a discontinued Phase II study in patients with major depressive disorder (MDD). Accordingly, OX2R-selective antagonists may be further preferred for combination with psychedelics in treating MDD and related mood disorders.

Since psychedelic drugs, particularly those activating the 5-HT2a receptor, also increase arousal and disturb sleep, combinations of orexin receptor antagonists with psychedelic drugs are synergistic, since the orexin receptor antagonist counteracts the sleep disturbance of the psychedelic while at the same time, the low to moderate dose psychedelic component enhances mood and cognition.

Psychedelic drugs, particularly those activating the 5-HT2a receptor, are also reported to improve mood and anxiety but can enhance arousal and disturb sleep. In some embodiments, the combination of an orexin receptor antagonist to maximally promote sleep and enhance SWS for learning along with a compound disclosed herein at the right dose are ideal for a broad spectrum of conditions where an orexin receptor antagonist alone or a psychedelic alone would not be efficacious.

In some embodiments, the present disclosure includes a method of treating a neuropsychiatric disorder comprising administering to a patient in need thereof a low to moderate dose of a serotonin receptor agonist or an NMDA receptor antagonist in combination with an orexin receptor antagonist prior to sleep. In some embodiments, the present methods enhance the natural neuroplasticity and cognition/memory enhancing effects of sleep. In some embodiments, methods disclosed herein allow a patient to get to sleep and maintain sleep that would typically be disrupted by the activating or arousing effects of psychedelic drugs (and where the psychedelic itself is enhancing neuroplasticity, mood, reducing anxiety, and improving state of mind and next-day function). In some embodiments, methods disclosed herein decrease the stressful effects of psychedelic drugs via the effect of the orexin receptor antagonist.

In some embodiments, combinations disclosed herein lead to multiple potential additive or synergistic effects, including: between the natural therapeutic effects of sleep for learning, memory, mood, anxiety, and stress; the effects of psychedelic drugs on these same parameters; and sleep incubation for prior day learning and training as well as sleep or dream incubation.

In some embodiments, the combination of an orexin receptor antagonist and a serotonin receptor agonist has reduced abuse liability compared to the serotonin receptor agonist alone, since increasing the dose of the serotonin receptor agonist in order to achieve greater psychedelic effects also increases the dose of the orexin receptor antagonist, promoting sleep and making the experience less desirable to recreational drug users.

### BRIEF DESCRIPTION OF THE DRAWINGS

FIG. 1 depicts nine line graphs showing acute effects of Psilocybin (Psi, A), Daridorexant (Dar, B) and their combination (Psi+Dar, C) in the first 4 h after dosing.

FIG. 2 depicts two bar graphs showing REM sleep onset latency (A) and NREM sleep onset latency (B) across vehicle (V), psilocybin 1 mg/kg (P1), psilocybin 3 mg/kg (P3), daridorexant 30 mg/kg (D30), daridorexant 100 mg/kg (D100), and combinations of daridorexant 100 mg/kg with psilocybin 1 mg/kg (D100+P1) and daridorexant 30 mg/kg with psilocybin 1 mg/kg (D30+P1).

FIG. 3 depicts three graphs illustrating percentage of time spent in Wake (A), NREM (B), and REM (C) stages across 22 h after dosing. Change from light cycle (passive) to dark cycle (active) is shown by vertical black line at 10 h 30 min after dosing. Psi 1 mg=psilocybin 1 mg/kg, Dar 30 mg=daridorexant 30 mg/kg, Dar 100 mg=daridorexant 100 mg/kg, Psi 1 mg+Dar 30 mg=combination of daridorexant 30 mg/kg with psilocybin 1 mg/kg, Psi 1 mg+Dar 100 mg=combination of daridorexant 100 mg/kg with psilocybin 1 mg/kg.

FIG. 4 depicts six graphs showing fragmentation of sleep measured as number of transitions between Wake and REM sleep (A), Wake and NREM sleep (B), and REM and NREM sleep (C). Change from light cycle (passive) to dark cycle (active) is shown by vertical black line at 10 h 30 min after dosing. Psi 1 mg=psilocybin 1 mg/kg, Dar 30 mg=daridorexant 30 mg/kg, Dar 100 mg=daridorexant 100 mg/kg, Psi 1 mg+Dar 30 mg=combination of daridorexant 30 mg/kg with psilocybin 1 mg/kg, Psi 1 mg+Dar 100 mg=combination of daridorexant 100 mg/kg with psilocybin 1 mg/kg.

FIG. 5 depicts a graph showing number of HTR observed in response to 4-HO-MET (0.32, 1, or 3.2 mg/kg, s.c.) administered 30 min after vehicle (0.5% methylcellulose) or daridorexant (100 mg/kg, p.o.) pretreatment.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

In the present specification, the term “therapeutically effective amount” means the amount of the subject compound that will elicit the biological or medical response of a tissue, system, animal or human that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in the treatment of depression.

“Hamilton Depression Scale 17-item (HAMD-17)” is a multiple item questionnaire used to provide an indication of depression, and is known to those skilled in the art. The questionnaire is designed for adults to rate the severity of depression by probing mood, feelings of guilt, suicide ideation, insomnia, work and interests, agitation or retardation, anxiety, weight loss, and somatic symptoms. Each item on the questionnaire is scored on a 3 or 5 point scale, depending on the item, and the total score is compared to the corresponding descriptor. A score of 0-7 is considered to be normal. Scores of 14 or higher indicate moderate, severe, or very severe depression. Assessment time is estimated at 20 minutes. (See Guy W. Clinical Global Impressions. In: ECDEU Assessment Manual for Psychopharmacology—Revised (DHEW publication number ADM 76-338). Rockville, MD: US Department of Health, Education, and Welfare. 1976; 218-222; Hamilton M. J. Neurol. Neurosurg. Psychiat. 1960; (23):56; Kind P. The EuroQol instrument: An index of health related quality of life. In: Spiker B, ed. Quality of Life and Pharmaco Economics in Clinical Trials. 2nd ed. Philadelphia, PA: Lippincott-Raven Publishers; 1996.)

“Hamilton Depression Scale 6-item (HAMD-6)” is a subscale derived from the 17-item Hamilton Rating Scale for Depression (HAMD-17). HAMD-6 includes the following items: depressed mood, guilt feelings, work and interests, psychomotor retardation, psychic anxiety, and general somatic symptoms (Bech et al. Acta Psychiatr Scand. 1975, 51(3): 161-70).

“Montgomery-Åsberg Depression Rating Scale (MADRS)” is a clinical rating scale for depression (Montgomery and Asberg, Br J Psychiatry. 1979; 134:382-389) and includes the following 10 items: 1) apparent sadness; 2) reported sadness; 3) inner tension; 4) reduced sleep; 5) reduced appetite; 6) concentration difficulties; 7) lassitude; 8) inability to feel; 9) pessimistic thoughts; and 10) suicidal thoughts. MADRS items are rated on a 0-6 continuum (0=no abnormality, 6=severe).

“Clinical Global Impression (CGI)” scale is a clinician-rated, 7-point scale that is designed to rate the severity of the subject's depression at the time of assessment using the Investigator's judgment and past experience with subjects who have the same disorder (i.e., depression with anxious distress). “Clinical Global Impression-Improvement (CGI-I)” is a clinician-rated, 7-point scale that is designed to rate the improvement in the subject's depression at the time of assessment, relative to the symptoms at Baseline. “Clinical Global Impression-Severity (CGI-S)” is an assessment of the current severity of the patient's disease.

“Shechan Disability Score (SDS)” is used to assess functional disability and improvement in workplace function has been demonstrated with antidepressant therapy in patients with MDD (Lee et al, J Affect Disord. 2018; 227:406-415). The SDS is well validated and widely accepted for assessing functional outcomes in patients with MDD (Weiller et al, Neuropsychiatr Dis Treat. 2017; 14:103-115). In a systematic review of studies that assessed functional outcomes, the authors suggested that improvements in function (SDS) should be considered for inclusion as co-endpoints with symptomatic assessments when evaluating treatments for MDD (Shechan et al, J Affect Disord. 2017; 215:299-313). Routine assessments of both symptoms and function could be helpful for minimizing residual effects that increase the risk for relapse or recurrence (Shechan et al, J Affect Disord. 2017; 215:299-313).

“Functional remission” as used herein refers to remission in functional impairment. Functional remission is defined as a SDS total score of 6 or less at endpoint. (Shechan et al. Human Psychopharmacology 2016, 31, 53-63).

“Karolinska Sleepiness Scale (KSS)” is a scale for evaluating subjective sleepiness (Kaida et al. Clinical Neurophysiology 2006, 117, 7, 1574-1581).

“The 12-Item Short-Form Health Survey (SH-12)” (Ware J E Jr et al. Med Care. 1996; 34(3):220-233) is a 12 item-questionnaire derived from the Medical Outcomes Study 36-Item Short-Form Health Survey (SF-36).

“Early improvement as measured by Hamilton Depression Scale,” as used herein, refers to a reduction from baseline in HAMD-17 total score of 20% or more.

A patient with “DMS-5 defined diagnosis of major depressive disorder” has 5 or more of the symptoms as described in Diagnostic and Statistical Manual of Mental Disorders, Fifth Edition (DSM-5) during a 2-week period and at least one of the symptoms is either 1) depressed mood or 2) loss of interest or pleasure. The symptoms as described in DMS-5 include the following: 1) depressed mood; 2) loss of interest or pleasure; 3) significant weight loss; 4) insomnia or hypersomnia; 5) psychomotor agitation or retardation; 6) fatigue or loss of energy; 7) feelings of worthlessness or excessive or inappropriate guilt; 8) diminished ability to think or concentrate or indecisiveness; and 9) recurrent thoughts of death.

“EuroQol-5 dimensions-5 level (EQ-5D-5L)” is a standardized instrument used as a measure of health outcome and measures 5 dimensions (mobility, self-care, usual activities, pain/discomfort, anxiety/depression), each of which has 5 potential responses. The responses record 5 levels of severity (no problems/slight problems/moderate problems/severe problems/extreme problems) within a particular EQ-5D dimension.

“Massachusetts General Hospital Sexual Functioning Index (MGH-SFI)” is described, for instance, in Labbate and Lare, 2001, Psychother Psychosom. 70(4):221-225. This is a patient facing questionnaire that quantifies sexual dysfunction into 5 functional domains (“interest in sex,” “sexual arousal,” “ability to achieve orgasm,” “ability to maintain erection” (males only), and “sexual satisfaction”). Patients rate each item using a 6-point scale ranging from 1 (good function) to 6 (poor function). High scores indicate poor sexual functioning.

The terms “addiction” and related disorders, “substance abuse disorder”, and “substance use disorder” are commonly used interchangeably by persons of ordinary skill in the art and in relevant literature.

“Subjective Opiate Withdrawal Scale” (SOWS-Handelsman)—The SOWS is a 16-item rating scale (0=not at all to 4=extremely) used by patients for measuring the severity of their opiate withdrawal symptoms. Total score 0 to 64

“Clinical Opiate Withdrawal Scale” (COWS) is used to assess a patient's level of opiate withdrawal, is a written instrument, administered by the investigator, and rates 11 common opiate withdrawal signs or symptoms. Total score 0 to 44.

### Methods

Provided herein, in part, is a method of treating a neuropsychiatric disorder in a patient in need thereof, comprising administering to the patient an effective amount of an orexin receptor antagonist and further comprising administering to the patient an effective amount of a serotonin receptor agonist or an NMDA receptor antagonist. In some embodiments, the present disclosure includes a method of improving sleep in a patient in need thereof, comprising administering to the patient an effective amount of an orexin receptor antagonist and further comprising administering to the patient an effective amount of a serotonin receptor agonist or an NMDA receptor antagonist. In some embodiments, a method of improving memory in a patient in need thereof, comprising administering to the patient an effective amount of an orexin receptor antagonist and further comprising administering to the patient an effective amount of a serotonin receptor agonist or an NMDA receptor antagonist. In some embodiments, a method of improving learning in a patient in need thereof, comprising administering to the patient an effective amount of an orexin receptor antagonist and further comprising administering to the patient an effective amount of a serotonin receptor agonist or an NMDA receptor antagonist.

### Orexin Receptor Antagonist

In some embodiments, the present disclosure includes a method of treating a neuropsychiatric disorder in a patient in need thereof, comprising administering to the patient an effective amount of an orexin receptor antagonist. In some embodiments, an orexin receptor antagonist is selected from the group consisting of suvorexant, lemborexant, seltorexant, daridorexant, filorexant, almorexant, and TS-142.

In some embodiments, an orexin receptor antagonist is suvorexant. In some embodiments, suvorexant is administered in a daily dose between 5 mg to 40 mg. In some embodiments, suvorexant is administered in a daily dose between 5 mg to 35 mg. In some embodiments, suvorexant is administered in a daily dose between 5 mg to 30 mg. In some embodiments, suvorexant is administered in a daily dose between 5 mg to 25 mg. In some embodiments, suvorexant is administered in a daily dose between 5 mg to 20 mg. In some embodiments, suvorexant is administered in a daily dose between 10 mg to 40 mg. In some embodiments, suvorexant is administered in a daily dose between 10 mg to 30 mg. In some embodiments, suvorexant is administered in a daily dose between 10 mg to 25 mg. In some embodiments, suvorexant is administered in a daily dose between 10 mg to 20 mg. In some embodiments, suvorexant is administered in a daily dose of about 5 mg. In some embodiments, suvorexant is administered in a daily dose of about 10 mg. In some embodiments, suvorexant is administered in a daily dose of about 15 mg. In some embodiments, suvorexant is administered in a daily dose of about 20 mg. In some embodiments, suvorexant is administered in a daily dose of about 25 mg. In some embodiments, suvorexant is administered in a daily dose of about 30 mg.

In some embodiments, an orexin receptor antagonist is lemborexant. In some embodiments, lemborexant is administered in a daily dose between 1 mg to 25 mg. In some embodiments, lemborexant is administered in a daily dose between 1 mg to 20 mg. In some embodiments, lemborexant is administered in a daily dose between 1 mg to 15 mg. In some embodiments, lemborexant is administered in a daily dose between 1 mg to 10 mg. In some embodiments, lemborexant is administered in a daily dose between 1 mg to 5 mg. In some embodiments, lemborexant is administered in a daily dose between 5 mg to 25 mg. In some embodiments, lemborexant is administered in a daily dose between 5 mg to 20 mg. In some embodiments, lemborexant is administered in a daily dose between 5 mg to 15 mg. In some embodiments, lemborexant is administered in a daily dose between 5 mg to 10 mg. In some embodiments, lemborexant is administered in a daily dose of about 1 mg. In some embodiments, lemborexant is administered in a daily dose of about 5 mg. In some embodiments, lemborexant is administered in a daily dose of about 10 mg. In some embodiments, lemborexant is administered in a daily dose of about 15 mg. In some embodiments, lemborexant is administered in a daily dose of about 20 mg.

In some embodiments, an orexin receptor antagonist is seltorexant. In some embodiments, seltorexant is administered in a daily dose between 1 mg to 45 mg. In some embodiments, seltorexant is administered in a daily dose between 1 mg to 40 mg. In some embodiments, seltorexant is administered in a daily dose between 1 mg to 30 mg. In some embodiments, seltorexant is administered in a daily dose between 1 mg to 25 mg. In some embodiments, seltorexant is administered in a daily dose between 1 mg to 25 mg. In some embodiments, seltorexant is administered in a daily dose between 1 mg to 15 mg. In some embodiments, seltorexant is administered in a daily dose between 5 mg to 45 mg. In some embodiments, seltorexant is administered in a daily dose between 5 mg to 35 mg. In some embodiments, seltorexant is administered in a daily dose between 5 mg to 30 mg. In some embodiments, seltorexant is administered in a daily dose between 5 mg to 25 mg. In some embodiments, seltorexant is administered in a daily dose between 5 mg to 20 mg. In some embodiments, seltorexant is administered in a daily dose between 10 mg to 35 mg. In some embodiments, seltorexant is administered in a daily dose between 10 mg to 30 mg. In some embodiments, seltorexant is administered in a daily dose between 10 mg to 25 mg. In some embodiments, seltorexant is administered in a daily dose between 10 mg to 20 mg. In some embodiments, seltorexant is administered in a daily dose between 15 mg to 20 mg. In some embodiments, seltorexant is administered in a daily dose of about 5 mg. In some embodiments, seltorexant is administered in a daily dose of about 10 mg. In some embodiments, seltorexant is administered in a daily dose of about 15 mg. In some embodiments, seltorexant is administered in a daily dose of about 20 mg. In some embodiments, seltorexant is administered in a daily dose of about 25 mg. In some embodiments, seltorexant is administered in a daily dose of about 30 mg. In some embodiments, seltorexant is administered in a daily dose of about 35 mg. In some embodiments, seltorexant is administered in a daily dose of about 40 mg. In some embodiments, seltorexant is administered in a daily dose of about 45 mg.

In some embodiments, an orexin receptor antagonist is daridorexant. In some embodiments, daridorexant is administered in a daily dose between 5 mg to 60 mg. In some embodiments, daridorexant is administered in a daily dose between 35 mg to 60 mg. In some embodiments, daridorexant is administered in a daily dose between 40 mg to 60 mg. In some embodiments, daridorexant is administered in a daily dose between 45 mg to 55 mg. In some embodiments, daridorexant is administered in a daily dose between 15 mg to 65 mg. In some embodiments, daridorexant is administered in a daily dose between 15 mg to 35 mg. In some embodiments, daridorexant is administered in a daily dose between 5 mg to 40 mg. In some embodiments, daridorexant is administered in a daily dose between 5 mg to 35 mg. In some embodiments, daridorexant is administered in a daily dose between 5 mg to 30 mg. In some embodiments, daridorexant is administered in a daily dose between 5 mg to 25 mg. In some embodiments, daridorexant is administered in a daily dose of about 50 mg.

In embodiments, an orexin receptor antagonist is filorexant.

In some embodiments, an orexin receptor antagonist is almorexant. In some embodiments, almorexant is administered in a daily dose between 50 mg to 500 mg. In some embodiments, almorexant is administered in a daily dose between 350 mg to 450 mg. In some embodiments, almorexant is administered in a daily dose between 375 mg to 425 mg. In some embodiments, almorexant is administered in a daily dose between 150 mg to 250 mg. In some embodiments, almorexant is administered in a daily dose between 175 mg to 225 mg. In some embodiments, almorexant is administered in a daily dose between 50 mg to 150 mg. In some embodiments, almorexant is administered in a daily dose between 75 mg to 125 mg. In some embodiments, almorexant is administered in a daily dose about 400 mg. In some embodiments, almorexant is administered in a daily dose about 300 mg. In some embodiments, almorexant is administered in a daily dose about 200 mg. In some embodiments, almorexant is administered in a daily dose about 100 mg.

In some embodiments, an orexin receptor antagonist is TS-142. In some embodiments, TS-142 is administered in a daily dose between 1 mg to 40 mg. In some embodiments, TS-142 is administered in a daily dose between 5 mg to 30 mg. In some embodiments, TS-142 is administered in a daily dose between 5 mg to 15 mg. In some embodiments, TS-142 is administered in a daily dose of about 5 mg. In some embodiments, TS-142 is administered in a daily dose of about 10 mg. In some embodiments, TS-142 is administered in a daily dose of about 30 mg.

In some embodiments, a method might include use of the combination daily for several weeks or 1-3 time per week.

The dose level of the psychedelic drugs as small to moderate are designed to limit activation and the orexin receptor antagonist would encourage and maintain sleep but without the cognitively negative effects of GABA-A agonists often used for sleep.

The combination of an orexin receptor antagonist+psychedelic-related drugs will allow for using sleep including dream sleep (REM sleep) and non-REM sleep as a time for enhanced therapeutic effect by enhancing neuroplasticity which occurs during sleep normally. Therefore, this combination medication regime especially when paired with behavioral approaches including dream incubation and learning of critical materials will be useful for a variety of psychiatric conditions and for general enhanced problem solving. Orexin receptor antagonists are known to increase both non-REM sleep and REM or dream sleep and sleep onset REM periods. This approach may also limit or eliminate the risk of substance use disorders or nonadherence to prescribed dose with psychedelics since talking a larger dose of this combined medicine will result in the person mainly being asleep longer or having disturbed sleep but without a recreational benefit.

### Serotonin Receptor Agonist

In some embodiments, the present disclosure includes a method of treating a neuropsychiatric disorder in a patient in need thereof, comprising administering to the patient an effective amount of Serotonin Receptor Agonist. In some embodiments, a serotonin receptor agonist is a serotonin 2A receptor agonist. In some embodiments, a serotonin receptor agonist is a serotonin 2C receptor agonist. In some embodiments, a serotonin receptor agonist activates the 5-HT2a receptor and optionally one or more additional serotonin receptors selected from the group consisting of 5-HT1a, 5-HT2b, and 5-HT2c.

In some embodiments, a serotonin receptor agonist activates the 5-HT2a receptor and is selected from a group consisting of a lysergic acid amide, a tryptamine, a phenethylamine, and an amphetamine. In some embodiments, a serotonin receptor agonist is a lysergic acid amide selected from a group consisting of lysergic acid diethylamide, lysergic acid 2,4-dimethylazetidide (LSZ), 6-ethyl-6-nor-lysergic acid diethylamide (ETH-LAD), 6-propyl-6-nor-Lysergic acid diethylamide (PRO-LAD), 1-Acetyl-N,N-diethyllysergamide (ALD-52), 1-propionyl-lysergic acid diethylamide (1P-LSD), N1-butyryl-lysergic acid diethylamide (1B-LSD), and N1-(cyclopropylmethanoyl)-lysergic acid diethylamide (1 cP-LSD). In some embodiments, a serotonin receptor agonist is a tryptamine selected from the group consisting of psilocybin, psilocin, N,N-dimethyltryptamine, 5-MeO—N,N-dimethyltryptamine, ibogaine, noribogaine, N-methyl-N-ethyltryptamine (MET), methylisopropyltryptamine (MIPT), diethyltryptamine (DET), diisopropyltryptamine (DIPT), dipropyltryptamine (DPT), ethylpropyltryptamine (EPT), 5-methoxy-methylisopropyltryptamine (5-MeO-MIPT), 5-methoxy-diisopropyltryptamine (5-MeO-DIPT), 5-methoxy-N-methyl-N-ethyltryptamine (5-MeO-MET), 5-methoxy-diethyltryptamine (5-MeO-DET), 4-hydroxy-N-methyl-N-cthyltryptamine (4-HO-MET), 4-hydroxy-methylisopropyltryptamine (4-HO-MIPT), 4-hydroxy-diisopropyltryptamine (4-HO-DIPT), 4-hydroxy-diethyltryptamine (4-HO-DET), 4-hydroxy-dipropyltryptamine (4-HO-DPT), 4-hydroxy-ethylpropyltryptamine (4-HO-EPT), 4-acetoxy-N-methyl-N-ethyltryptamine (4-AcO-MET), 4-acetoxy-methylisopropyltryptamine (4-AcO-MIPT), 4-acetoxy-diisopropyltryptamine (4-AcO-DIPT), 4-acetoxy-diethyltryptamine (4-AcO-DET), 4-acetoxy-dipropyltryptamine (4-AcO-DPT), 4-acetoxy-ethylpropyltryptamine (4-AcO-EPT), 4-acetoxy-dimethyltryptamine (4-AcO-DMT), alpha-methyltryptamine, and alpha-ethyltryptamine. In some embodiments, a serotonin receptor agonist is a phenethylamine selected from the group consisting of mescaline, escaline, proscaline, methallylescaline, 4-bromo-2,5-dimethoxylpenethylamine (2C-B), 2,5-dimethoxy-4-methylphenethylamine (2C-D), 2-(4-Ethyl-2,5-dimethoxyphenyl)ethanamine (2C-E), 2-(2,5-Dimethoxy-4-propylphenyl)ethan-1-amine (2C-P), 2-[4-(Ethylsulfanyl)-2,5-dimethoxyphenyl]ethan-1-amine (2C-T-2), and 2-[2,5-Dimethoxy-4-(propylsulfanyl)phenyl]ethan-1-amine (2C-T-7). In some embodiments, serotonin receptor agonist is an amphetamine selected from the group consisting of 2,5-dimethoxy-4-methylamphetamine (DOM), 2,5-dimethoxy-4-bromoamphetamine (DOB), 2,5-dimethoxy-4-chloroamphetamine (DOC,) 2,5-dimethoxy-4-iodoamphetamine (DOI), 2,5-dimethoxy-4-ethylamphetamine (DOET), and 2,5-Dimethoxy-4-propylamphetamine (DOPR). In some embodiments, a serotonin receptor agonist is selected from the group consisting of lysergic acid diethylamide, psilocybin, psilocin, 4-AcO-DMT, N,N-dimethyltryptamine, 5-MeO—N,N-dimethyltryptamine, mescaline, 2C-B, and 2C-E. In some embodiments, a serotonin receptor agonist is selected from a lysergic acid amide, ergoline, mescaline, psilocybin, bufotenin, ibogaine, psilotsin, N,N-dimethyltryptamine, and 5-MeO—N,N-dimethyltryptamine. In some embodiments, a serotonin receptor agonist is lysergic acid diethylamide.

In some embodiments, a serotonin receptor agonist is represented by

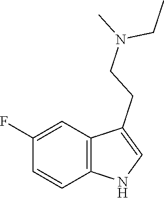 [SMILES: CCN(C)CCc1c[nH]c2ccc(F)cc12]

or a pharmaceutically acceptable salt thereof.

In some embodiments, a serotonin receptor agonist is represented by

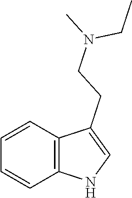

or a pharmaceutically acceptable salt thereof.

In some embodiments, a serotonin receptor agonist is psilocybin. In some embodiments, psilocybin is crystalline psilocybin. In some embodiments, crystalline psilocybin is disclosed in U.S. Pat. No. 10,519,175. In some embodiments, crystalline psilocybin is a polymorph characterized by peaks in an XRPD diffractogram at 11.5, 12.0, 14.5, 17.5, and 19.7°2θ±0.1°2θ. In some embodiments, crystalline psilocybin is a polymorph characterized by a peak at 17.5°2θ±0.1°2θ has a relative intensity compared to the peak at 14.5°2θ±0.1°2θ of at least 5%. In some embodiments, crystalline psilocybin is a polymorph characterized by a peak at 17.5°2θ±0.1°2θ has a relative intensity compared to the peak at 14.5°2θ±0.1°2θ of at least 9%. In some embodiments, crystalline psilocybin is a polymorph characterized by a peak at 10.1°2θ±0.1°2θ is absent or substantially absent. In some embodiments, crystalline psilocybin is a polymorph characterized by a peak at 10.1°2θ±0.1°2θ has a relative intensity compared to the peak at 14.5°2θ±0.1°2θ of less than 2%. In some embodiments, crystalline psilocybin is a polymorph characterized by a peak at 10.1°2θ±0.1°2θ has a relative intensity compared to the peak at 14.5°2θ±0.1°2θ of less than 1%. psilocybin is a polymorph, wherein a peak at 10.1°2θ±0.1°2θ is not detectable in the XRPD diffractogram. In some embodiments, an oral dosage form of crystalline psilocybin comprises 1 mg to 40 mg of crystalline psilocybin in the form Polymorph A. In some embodiments, an oral dosage form of crystalline psilocybin comprises 5 mg of crystalline psilocybin in the form Polymorph A. In some embodiments, an oral dosage form of crystalline psilocybin comprises 10 mg of crystalline psilocybin in the form Polymorph A. In some embodiments, an oral dosage form of crystalline psilocybin comprises 25 mg of crystalline psilocybin in the form Polymorph A.

In some embodiments, an oral dosage form of crystalline psilocybin comprises silicified microcrystalline cellulose with a particle size range from about 45 to 150 microns.

In some embodiments, an oral dosage form of crystalline psilocybin comprises a mixture of two silicified microcrystalline cellulose variants wherein the first variant has a particle size from about 45 to 80 microns and the second variant has a particle size of about 90 to 150 microns.

### NMDA Receptor Antagonist

In some embodiments, an NMDA receptor antagonist is selected from the group consisting of ketamine, (R)-ketamine, (S)-ketamine and a compound of selected from the group consisting of

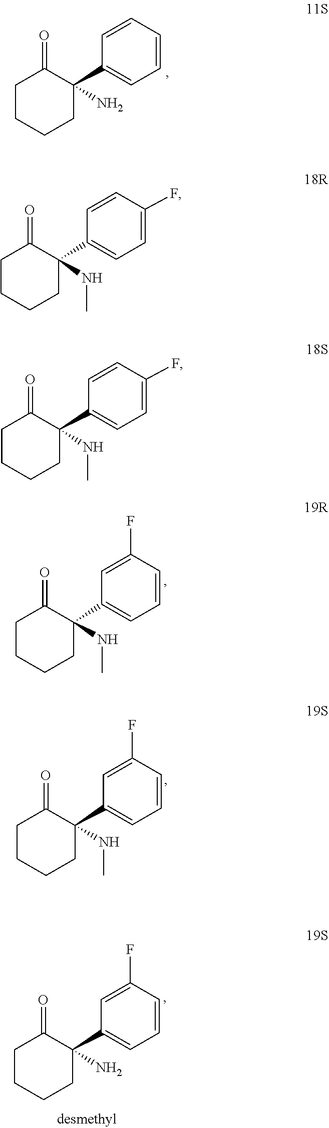 [SMILES: N[C@]1(c2ccccc2)CCCCC1=O ; CN[C@@]1(c2ccc(F)cc2)CCCCC1=O ; CN[C@]1(c2ccc(F)cc2)CCCCC1=O ; CN[C@@]1(c2cccc(F)c2)CCCCC1=O ; CN[C@]1(c2cccc(F)c2)CCCCC1=O ; N[C@]1(c2cccc(F)c2)CCCCC1=O]

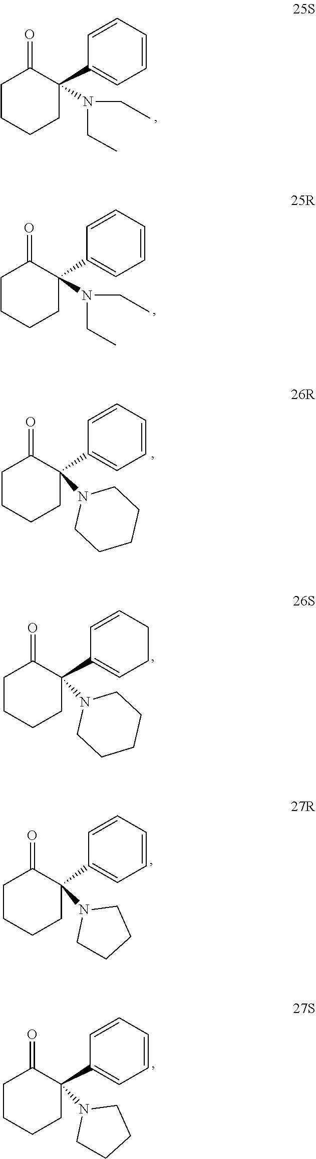 [SMILES: CCN(CC)[C@]1(c2ccccc2)CCCCC1=O ; CCN(CC)[C@@]1(c2ccccc2)CCCCC1=O ; O=C1CCCC[C@]1(c1ccccc1)N1CCCCC1 ; O=C1CCCC[C@@]1(C1=CCCC=C1)N1CCCCC1 ; O=C1CCCC[C@]1(c1ccccc1)N1CCCC1 ; O=C1CCCC[C@@]1(c1ccccc1)N1CCCC1]

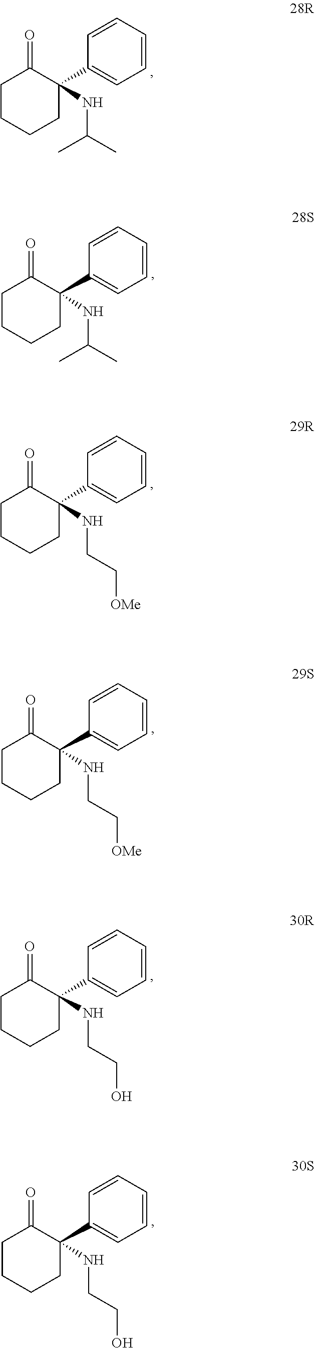 [SMILES: CC(C)N[C@@]1(c2ccccc2)CCCCC1=O ; CC(C)N[C@]1(c2ccccc2)CCCCC1=O ; COCCN[C@@]1(c2ccccc2)CCCCC1=O ; COCCN[C@]1(c2ccccc2)CCCCC1=O ; O=C1CCCC[C@@]1(NCCO)c1ccccc1 ; O=C1CCCC[C@]1(NCCO)c1ccccc1]

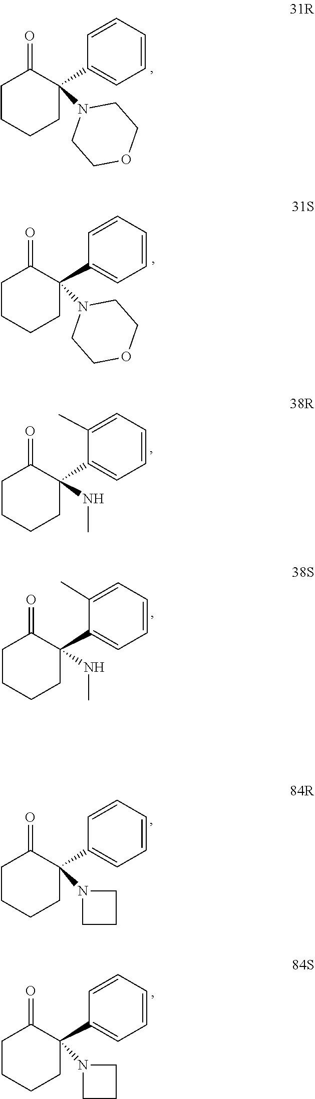 [SMILES: O=C1CCCC[C@]1(c1ccccc1)N1CCOCC1 ; O=C1CCCC[C@@]1(c1ccccc1)N1CCOCC1 ; CN[C@@]1(c2ccccc2C)CCCCC1=O ; CN[C@]1(c2ccccc2C)CCCCC1=O ; O=C1CCCC[C@]1(c1ccccc1)N1CCC1 ; O=C1CCCC[C@@]1(c1ccccc1)N1CCC1]

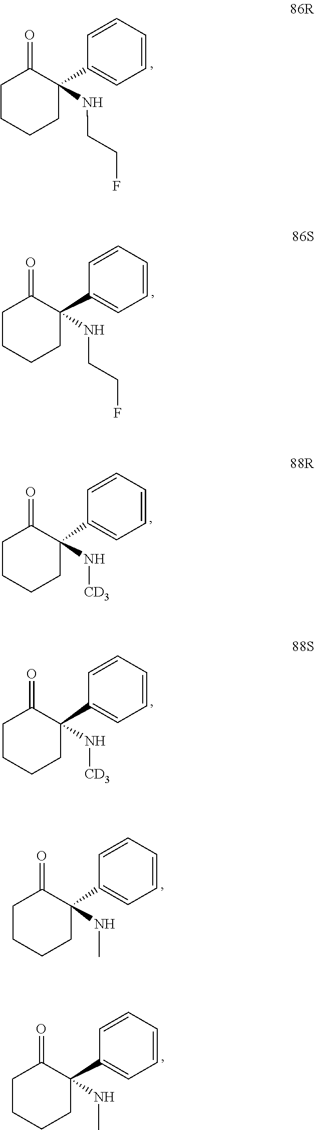 [SMILES: O=C1CCCC[C@@]1(NCCF)c1ccccc1 ; O=C1CCCC[C@]1(NCCF)c1ccccc1 ; [2H]C([2H])([2H])N[C@@]1(c2ccccc2)CCCCC1=O ; [2H]C([2H])([2H])N[C@]1(c2ccccc2)CCCCC1=O ; CN[C@@]1(c2ccccc2)CCCCC1=O ; CN[C@]1(c2ccccc2)CCCCC1=O]

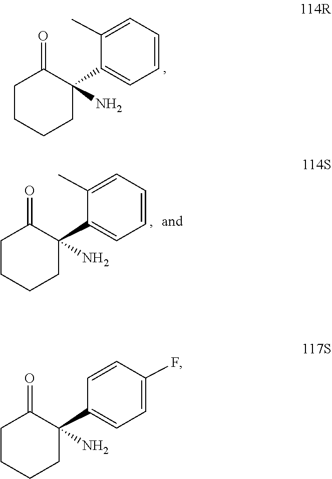

or a pharmaceutically acceptable salt thereof.

### Conditions

In some embodiments, methods described herein are useful in the treatment of a neuropsychiatric disorder. In some embodiments, a neuropsychiatric disorder is a mood disorder. In some embodiments, a mood disorder is depression.

In some embodiments, depression is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, prolonged or pathological grief, and a depressive disorder due to another medical condition. In some embodiments, depression is depression with ruminations. In some embodiments, depression is depression with anhedonia. In some embodiments, depression is depression with anxiety. In some embodiments, depression is depression with sleep disturbance.

In some embodiments, a mood disorder is a substance-related disorder. In some embodiments, a mood disorder is a substance-use disorder. In some embodiments of the methods disclosed herein, the substance use disorder includes misuse, abuse, or dependence or any habit-forming disorder. In some embodiments,

In some embodiments, substance use disorder, also known as chemical addiction, substance dependence, or addiction to a substance is selected from the group consisting of addiction including, but not limited to, stimulants (e.g., cocaine, amphetamines, methamphetamines, methylphenidate, and related stimulants), opioids (e.g., heroin, fentanyl, codeine, hydrocodone, and related opioid drugs), nicotine, alcohol, prescription medications (e.g., sedative-hypnotic drugs, medications prescribed for pain management such as oxycodone, hydrocodone and other non-opioid pain medicines), naturally-occurring plant-derived drugs (e.g. marijuana, tobacco, and the addictive agents therein) and synthetic drugs.

Addiction to an activity, also known as physical addiction, behavioral or behavioural addiction, soft addiction, process addiction or non-substance-related addiction is addiction to activities including, but not limited to, eating, food, exercise, gambling, sex, viewing of pornography, use of computers, use of the internet, playing video games, work, spiritual obsession, cutting (self-harm), travel or shopping.

In some embodiments, criteria for classifying a substance use disorder includes mild, moderate or severe. In some embodiments of the methods disclosed herein, the substance use disorder is selected from a mild substance use disorder, a moderate substance use disorder or a severe substance use disorder. In some embodiments, the substance use disorder is a mild substance use disorder. In some embodiments, the substance use disorder is a moderate substance use disorder. In some embodiments, the substance use disorder is a severe substance use disorder.

In some embodiments, a mood disorder is selected from the group consisting of obsessive-compulsive and related disorders, trauma- and stressor-related disorders, feeding and eating disorders, borderline personality disorder, attention-deficit/hyperactivity disorder, and autism spectrum disorder. In some embodiments, a mood disorder is an obsessive-compulsive disorder. In some embodiments, a mood disorder is post-traumatic stress disorder. In some embodiments, a mood disorder is attention-deficit/hyperactivity disorder.

In some embodiments, a mood disorder is prolonged or pathological grief.

In some embodiments, a neuropsychiatric disorder is a neurocognitive disorder. In some embodiments, a neurocognitive disorder is selected from the group consisting of neurocognitive disorder, mild neurocognitive disorder, major or mild neurocognitive disorder due to Alzheimer's disease, major or mild frontotemporal neurocognitive disorder, major or mild neurocognitive disorder with Lewy bodies, major or mild vascular neurocognitive disorder, major or mild neurocognitive disorder due to traumatic brain injury, substance/medication-induced major or mild neurocognitive disorder, major or mild neurocognitive disorder due to HIV infection, major or mild neurocognitive disorder due to prion disease, major or mild neurocognitive disorder due to Parkinson's disease, major or mild neurocognitive disorder due to Huntington's disease, major or mild neurocognitive disorder due to another medical condition, and major or mild neurocognitive disorder due to multiple etiologies. In some embodiments, a neurocognitive disorder is Alzheimer's disease.

In some embodiments, a neuropsychiatric disorder is a mild traumatic head injury.

### Treatment

In some embodiments, the present disclosure includes a method of treating a neuropsychiatric disorder in a patient in need thereof, comprising administering to the patient an effective amount of an orexin receptor antagonist and further comprising administering to the patient an effective amount of a serotonin receptor agonist or an NMDA receptor antagonist, wherein the orexin and a serotonin receptor agonist or an NMDA receptor antagonist and administered simultaneously. In some embodiments, methods described herein comprise administering to a patient an effective amount of an orexin receptor antagonist prior to administering to the patient an effective amount of a serotonin receptor agonist or an NMDA receptor antagonist. In some embodiments, methods described herein comprise administering to a patient an effective amount of an orexin receptor antagonist after administering to the patient an effective amount of a serotonin receptor agonist or an NMDA receptor antagonist. In some embodiments, methods described herein comprise administering to a patient an effective amount of an orexin receptor antagonist, wherein the patient had previously received an effective amount of a serotonin receptor agonist or an NMDA receptor antagonist. In some embodiments, methods described herein comprise administering to a patient an effective amount of an effective amount of a serotonin receptor agonist or an NMDA receptor antagonist, wherein the patient had previously received an effective amount of an orexin receptor antagonist.

In some embodiments, methods described herein comprise administering to a patient an effective amount of an orexin receptor antagonist and/or a serotonin receptor agonist or an NMDA receptor antagonist 0-120 minutes before bedtime. In some embodiments, methods described herein comprise administering to a patient an effective amount of an orexin receptor antagonist and/or a serotonin receptor agonist or an NMDA receptor antagonist 10-120 minutes before bedtime. In some embodiments, methods described herein comprise administering to a patient an effective amount of an orexin receptor antagonist and/or a serotonin receptor agonist or an NMDA receptor antagonist 30-120 minutes before bedtime. In some embodiments, methods described herein comprise administering to a patient an effective amount of an orexin receptor antagonist and/or a serotonin receptor agonist or an NMDA receptor antagonist 60-120 minutes before bedtime. In some embodiments, methods described herein comprise administering to a patient an effective amount of an orexin receptor antagonist and/or a serotonin receptor agonist or an NMDA receptor antagonist 90-120 minutes before bedtime. In some embodiments, methods described herein comprise administering to a patient an effective amount of an orexin receptor antagonist and/or a serotonin receptor agonist or an NMDA receptor antagonist 0-90 minutes before bedtime. In some embodiments, methods described herein comprise administering to a patient an effective amount of an orexin receptor antagonist and/or a serotonin receptor agonist or an NMDA receptor antagonist 0-60 minutes before bedtime. In some embodiments, methods described herein comprise administering to a patient an effective amount of an orexin receptor antagonist and/or a serotonin receptor agonist or an NMDA receptor antagonist 0-30 minutes before bedtime. In some embodiments, methods described herein comprise administering to a patient an effective amount of an orexin receptor antagonist and/or a serotonin receptor agonist or an NMDA receptor antagonist 0-10 minutes before bedtime.

In some embodiments, methods described herein comprise administering to a patient an effective amount of an orexin receptor antagonist and/or a serotonin receptor agonist or an NMDA receptor antagonist at bedtime. In some embodiments, methods described herein comprise administering to a patient an effective amount of an orexin receptor antagonist and/or a serotonin receptor agonist or an NMDA receptor antagonist about 10 minutes before bedtime. In some embodiments, methods described herein comprise administering to a patient an effective amount of an orexin receptor antagonist and/or a serotonin receptor agonist or an NMDA receptor antagonist about 30 minutes before bedtime. In some embodiments, methods described herein comprise administering to a patient an effective amount of an orexin receptor antagonist and/or a serotonin receptor agonist or an NMDA receptor antagonist about 60 minutes before bedtime. In some embodiments, methods described herein comprise administering to a patient an effective amount of an orexin receptor antagonist and/or a serotonin receptor agonist or an NMDA receptor antagonist about 90 minutes before bedtime. In some embodiments, methods described herein comprise administering to a patient an effective amount of an orexin receptor antagonist and/or a serotonin receptor agonist or an NMDA receptor antagonist about 120 minutes before bedtime.

In some embodiments, methods described herein comprise administering to a patient an effective amount of an orexin receptor antagonist and/or a serotonin receptor agonist or an NMDA receptor antagonist once daily. In some embodiments, methods described herein comprise administering to a patient an effective amount of an orexin receptor antagonist and/or a serotonin receptor agonist or an NMDA receptor antagonist once weekly. In some embodiments, methods described herein comprise administering to a patient an effective amount of an orexin receptor antagonist and/or a serotonin receptor agonist or an NMDA receptor antagonist twice a week. In some embodiments, methods described herein comprise administering to a patient an effective amount of an orexin receptor antagonist and/or a serotonin receptor agonist or an NMDA receptor antagonist three times a week. In some embodiments, methods described herein comprise administering to a patient an effective amount of an orexin receptor antagonist and/or a serotonin receptor agonist or an NMDA receptor antagonist four times a week. In some embodiments, a combination is administered in clinic. In some embodiments, a combination is administered at home.

In some embodiments, methods of the present disclosure further comprise behavioral approach therapy. In some embodiments, behavioral approach therapy is selected from the group consisting of dream incubation, preparation critical learning, and prior day cognitive task performance.

Unless otherwise indicated, a subject can be a mammal, a primate, a monkey or a human. In certain embodiments of the methods provided herein, the subject is a human subject.

In some embodiments, the human subject is a female human subject. In some embodiments, the human subject is a male human subject. In some embodiments, the human subject is a female or male human subject.

In some embodiments, the age of the human subject is 45 years or older, 50 years or older, or 55 years or old, or 60 years or older, or 65 years or older, or 70 years or older at baseline. In another embodiment, the age of the patient is 50 years or older. In another embodiment, the age of the patient is less than 50 years old.

In some embodiments, after 8 weeks of administration, the patient has a 50% or greater improvement relative to the baseline in a score on a scale selected from the group consisting of MADRS, HDRS, QIDS, HAM-D6, and adjusted HRDS17.

In some embodiments, after 8 weeks of administration, the patient has a 50% or greater improvement relative to the baseline in a score on opioid craving VAS, high VAS, SOWS—Handelsman or COWS. In some embodiments, after 8 weeks of administration, the patient has a 50% or greater improvement relative to the baseline in a score on SOWS—Handelsman. In some embodiments, after 8 weeks of administration, the patient has a 50% or greater reduction in opioid craving VAS.

In some embodiments, after 8 weeks of administration, the patient has a 50% or greater improvement relative to the baseline in a score on the Ruminative Response Scale.

In some embodiments, after 8 weeks of administration, the patient has a 50% or greater improvement relative to the baseline in a score on the Snaith Hamilton Anhedonia Pleasure Scale.

In some embodiments, after 8 weeks of administration, the patient has a 50% or greater improvement relative to the baseline in a score on the Karolinska Sleepiness Scale.

In some embodiments, after 8 weeks of administration, the patient has a 50% or greater improvement relative to the baseline in a score on the Digit Symbol Substitution Test.

In some embodiments, after 8 weeks of administration, the patient has a 50% or greater improvement relative to the baseline in a score on the Prolonged Grief Disorder-13.

In some embodiments, after 8 weeks of administration, the patient has a 50% or greater improvement in a HAM-A score relative to the baseline.

In some embodiments, after 8 weeks of administration, the patient has a 50% or greater improvement in a score on the Liebowitz Social Anxiety Scale relative to the baseline.

In some embodiments, wherein after 8 weeks of administration, the patient has a 50% or greater improvement relative to the baseline in a score on the Yale-Brown Obsessive Compulsive Scale (YBOCS).

In some embodiments, after 8 weeks of administration, the patient has a 50% or greater improvement relative to the baseline in a score on the Clinician-Administered PTSD Scale for DSM-5 (CAPS-5).

In some embodiments, after 8 weeks of administration, the patient has a 50% or greater improvement relative to the baseline in a score on a scale selected from the group consisting of Conners' Adult ADHD Rating Scale-Investigator Rated, AAQOL-29, and cognitive testing (e.g., CogState or CANTAB).

In some embodiments, methods described herein provide improvement in at least one symptom selected from the group consisting of sadness or lethargy or lassitude, depressed mood, inability to feel, anxious worried feelings, fears, feeling tense, feeling restlessness, diminished interest in all or nearly all activities, difficulty initiating activities, significant increased or decreased appetite leading to weight gain or weight loss, insomnia, sleep disturbance, irritability, fatigue, feelings of worthlessness or low self-esteem, strongly held negative beliefs or pessimistic thoughts about self, others or world, feelings of helplessness, inability to concentrate or distractibility, suicidality, feelings of guilt, memory complaints, difficulty experiencing positive feelings, feeling cut off or distant from people, hypervigilance, risk taking behavior, avoidance of thoughts about a stressful or traumatic event, pains and aches, ruminations and obsessive thoughts, compulsive behaviors, talking to people you don't know well or strangers, being center of attention, disturbing intrusive thoughts, can't get through week without drug use, guilty about drug use, problems with friends or family due to drug use, and withdrawal symptoms due to drug use.

In some embodiments, after 8 weeks of administration, the patient has a 50% or greater improvement relative to the baseline in a score on a scale selected from the group consisting of WASO, Total Sleep Time (TST), Sleep Quality VAS, Leeds Sleep Evaluation Questionnaire (LSEQ) and Latency to Persistent.

In some embodiments, after 8 weeks of administration, the patient has a 50% or greater improvement relative to the baseline in a score on a scale selected from the group consisting of LPS, AAQOL-29, sleep efficiency (SE), Leeds Sleep Evaluation Questionnaire (LSEQ), Polysomnogram, sleep diary.

In some embodiments, after 8 weeks of administration, the patient has a 50% or greater improvement relative to the baseline in a score on Laukes Emotional Intensity Scale (LEIS) or Emotional Breakthrough Inventory.

### EXAMPLES

### Example 1: Study of Safety and Efficacy of a Combination Comprising an Orexin Receptor Antagonist and a Serotonin Receptor Agonist or an NMDA Receptor Antagonist in Adults with a Neuropsychiatric Disorder

A study evaluating the safety and efficacy of a combination comprising an orexin receptor antagonist and a serotonin receptor agonist or an NMDA receptor antagonist in adults with a neuropsychiatric disorder is conducted. Subjects initially receive behavioral therapy such as dream incubation or prior day cognitive task or other behavioral training or therapy.

Following behavioral therapy, subjects are randomized into one of the following groups: 1-3 times per week for 2-6 weeks or daily over for 2-6 weeks. Potentially less frequent maintenance therapy following response. The following combinations are evaluated:
Orexin receptor antagonist+psychedelic related drug
Orexin receptor antagonist+placebo
Diphenhydramine+psychedelic

Next morning following administration of agents, dreams are recorded, and integration therapy administer to consolidate improvement. A randomized withdrawal design may also be employed for assessing maintenance:

Patients/subjects who are responsive to a combination (e.g., that have a 50% or >response to acute treatment) are blindly randomized to continue the treatment or to receive placebo. The patients/subjects are then monitored over time to assess time to relapse or other negative outcome (e.g., a certain % worsening).

EEG or phone-based sleep monitoring may be used to help with dose adjustment and record sleep talking or other sleep behavior. Awakening following a REM period may be considered to help with memory consolidation of dreams. Changes in sleep may also be used as a biomarker to trigger the need for retreatment with the therapy (e.g., worsening sleep pattern).

### Example 2: Effects of a Combination of Daridorexant and Psilocybin on Sleep

### Background

The aim of this study was to investigate the effects of daridorexant, psilocybin, and the combination of the two compounds, on sleep-wake behaviour in Sprague Dawley rats, across the circadian cycle. Daridorexant is a dual orexin receptor antagonist used for treating insomnia. Psilocybin is a 5-HT2A receptor agonist with psychedelic properties and relatively short half-life. The study was conducted at Transpharmation, UK, under Study IDs EEG079a and EEG079c.

### Methods

### Animals

8 male Sprague Dawley rats (Charles River, UK) were used in the study. Animals were housed in groups of 2-3 with standard conditions. Average weight at start of dosing was 433-517 g, corresponding to ˜ 14 weeks old.

### Surgery

All procedures were approved by the United Kingdom Home Office Animals (Scientific Procedures) Act 1986.

The animals were anaesthetised with isoflurane (2-5% in oxygen at 1 L/min) throughout the procedure. The scalp, neck, and right flank were cleared of hair using clippers and cleaned using diluted Hibitane (50% (v/v) in water). A non-steroidal anti-inflammatory agent was administered (Carprofen 5 mg/kg, subcutaneously (s.c.)) and the animals placed on a homeothermic blanket (37° C.) with the head fixed in a stereotaxic frame. Incisions were made in the scalp and neck, as well as in the right flank. Blunt dissection of the abdominal muscle was then made to gain access to the peritoneal cavity. A radio transmitter (HD-S02, Data Sciences International) was implanted in the peritoneal cavity and secured to the muscle wall with non-absorbable sutures. The wires of the transmitter were passed using a 16 G needle through the muscle wall and then sub-dermally to the scalp to act as EEG/EMG electrodes. The scalp was cleared of connective tissue and two craniotomies were made with a trepanning drill (fronto-parietal coordinates; Bregma+2 mm anterior, midline+1.0 mm lateral, and Lambda 0 mm, +1.5 mm lateral). The positive EEG electrode was attached to the anterior craniotomy and the negative EEG electrode to the posterior craniotomy. Both electrodes were secured in place using a suitable adhesive agent (cyanoacrylate gel, RS components). A second set of electrodes were sutured into the nuchal muscle to act as EMG electrodes. All incisions were closed using absorbable suture.

All animals were allowed to recover in a warm environment and given 5 mL of saline s.c. Animals were kept singly in the cage until the next day, then pair housed for the duration of post-surgical recovery (minimum 7 days). During the recovery period the rats received 5 days of standard post-operative care with no further experimental procedures until the pre-operative body weight was regained. The animals were maintained on a 12/12 h light-dark cycle (08:00 am lights on) after the surgery.

### Testing

On study days, at ˜30 min after lights-on, animals were placed in recording boxes similar to their home cages and data acquisition was begun for confirmation of signal quality. After 30 min, each animal was briefly removed from its box to be dosed (see schedule below). Animals were immediately put back into the recording boxes, the experimenter left the room, and recordings continued uninterrupted for ˜23 h. At the end of the recording session, the transmitters were turned off and the animals were returned to their home cages. Animals received treatment conditions in a pseudo randomised cross-over fashion with a minimum period of 7 days between doses.

### Test Compound Details

Daridorexant was administered p.o. at a volume of 5 mL/kg with 0.5% methylcellulose vehicle (Vehicle 1), at doses of 30 mg/kg or 100 mg/kg. Psilocybin was administered s.c. at a volume of 3 mL/kg with saline vehicle (Vehicle 2), at doses of 0.5 mg/kg, 1 mg/kg, or 3 mg/kg.

### Treatment Schedule

| day | box | id | treatment | day | box | id | treatment |
|---|---|---|---|---|---|---|---|
| 1 | 1 | 1 | Vehicle1_Vehicle2 | 2 | 1 | 1 | Vehicle1_Psilo3 mg |
| 1 | 2 | 2 | Vehicle1_Psilo3 mg | 2 | 2 | 2 | Dar100 mg_Vehicle2 |
| 1 | 3 | 3 | Dar100 mg_Vehicle2 | 2 | 3 | 3 | Dar100 mg_Psilo3 mg |
| 1 | 4 | 4 | Dar100 mg_Psilo3 mg | 2 | 4 | 4 | Vehicle1_Vehicle2 |
| 1 | 5 | 5 | Vehicle1_Vehicle2 | 2 | 5 | 5 | Vehicle1_Psilo3 mg |
| 1 | 6 | 6 | Vehicle1_Psilo3 mg | 2 | 6 | 6 | Dar100 mg_Vehicle2 |
| 1 | 7 | 7 | Dar100 mg_Vehicle2 | 2 | 7 | 7 | Dar100 mg_Psilo3 mg |
| 1 | 8 | 8 | Dar100 mg_Psilo3 mg | 2 | 8 | 8 | Vehicle1_Vehicle2 |
| 3 | 1 | 1 | Dar100 mg_Vehicle2 | 4 | 1 | 1 | Dar100 mg_Psilo3 mg |
| 3 | 2 | 2 | Dar100 mg_Psilo3 mg | 4 | 2 | 2 | Vehicle1_Vehicle2 |
| 3 | 3 | 3 | Vehicle1_Vehicle2 | 4 | 3 | 3 | Vehicle1_Psilo3 mg |
| 3 | 4 | 4 | Vehicle1_Psilo3 mg | 4 | 4 | 4 | Dar100 mg_Vehicle2 |
| 3 | 5 | 5 | Dar100 mg_Vehicle2 | 4 | 5 | 5 | Dar100 mg_Psilo3 mg |
| 3 | 6 | 6 | Vehicle1_Vehicle2 | 4 | 6 | 6 | Dar100 mg_Psilo3 mg |
| 3 | 7 | 7 | Vehicle1_Vehicle2 | 4 | 7 | 7 | Vehicle1_Psilo3 mg |
| 3 | 8 | 8 | Vehicle1_Psilo3 mg | 4 | 8 | 8 | Dar100 mg_Vehicle2 |
| 5 | 1 | 1 | Dar100 mg_ Psilo1 mg | 6 | 1 | 1 | Dar30 mg_Psilo1 mg |
| 5 | 2 | 2 | Dar100 mg_ Psilo1 mg | 6 | 2 | 2 | Vehicle1_ Psilo1 mg |
| 5 | 3 | 3 | Dar100 mg_Psilo0.5 mg | 6 | 3 | 3 | Dar30 mg_Psilo1 mg |
| 5 | 4 | 4 | Dar100 mg_Psilo0.5 mg | 6 | 4 | 4 | Dar30 mg_Psilo1 mg |
| 5 | 5 | 5 | Dar100 mg_ Psilo1 mg | 6 | 5 | 5 | Vehicle1_Vehicle2 |
| 5 | 6 | 6 | Dar100 mg_ Psilo1 mg | 6 | 6 | 6 | Vehicle1_ Psilo1 mg |
| 5 | 7 | 7 | Dar100 mg_Psilo0.5 mg | 6 | 7 | 7 | Dar30 mg_Vehicle2 |
| 5 | 8 | 8 | Dar100 mg_Psilo0.5 mg | 6 | 8 | 8 | Vehicle1_ Psilo1 mg |

| day | box | id | treat | day | box | id | treat |
|---|---|---|---|---|---|---|---|
| 7 | 1 | 1 | Dar30 mg_Vehicle2 | 8 | 1 | 1 | Vehicle1_ Psilo1 mg |
| 7 | 2 | 2 | Vehicle1_Vehicle2 | 8 | 2 | 2 | Dar30 mg_Psilo 1 mg |
| 7 | 3 | 3 | Vehicle1_Psilo1 mg | 8 | 3 | 3 | Dar30 mg_Vehicle2 |
| 7 | 4 | 4 | Dar30 mg_Vehicle2 | 8 | 4 | 4 | Vehicle1_Vehicle2 |
| 7 | 5 | 5 | Dar30 mg_Psilo1 mg | 8 | 5 | 5 | Vehicle1_ Psilo1 mg |
| 7 | 6 | 6 | Vehicle1_Vehicle2 | 8 | 6 | 6 | Dar30 mg_Vehicle2 |
| 7 | 7 | 7 | Vehicle1_Psilo1 mg | 8 | 7 | 7 | Dar30 mg_Psilo1 mg |
| 7 | 8 | 8 | Vehicle1_Vehicle2 | 8 | 8 | 8 | Dar30 mg_Psilo1 mg |

### Data Analysis

EEG, EMG, and locomotor activity data were acquired for ˜23 h post dosing using intraperitoneal HD-S02 transmitters (DSI, New Brighton, MN) and Spike2 software (CED, Cambridge UK). EEG/EMG signals were amplified, analogue filtered (0.5-100 Hz), digitized (256 Hz), and then digitally filtered (EEG: 0.5-100 Hz and EMG: 70-100 Hz).

The recordings were automatically sleep-scored in 10 second epochs as wake, non-REM (NREM) sleep, or REM sleep using custom software that used EMG and EEG spectral components for sleep classification. The software was independently validated by experienced human sleep scorers. Non-REM sleep latency was defined as the time to the first 6 consecutive epochs scored as non-REM sleep, allowing 1 epoch of another type to interrupt the sequence. For REM sleep, the criterion was 3 consecutive epochs. Mean plots were computed by averaging values of interest in predefined time windows across all animals that received the same treatment. Standard error of mean (S.E.M.) is shown in figures in addition to mean values.

### Results

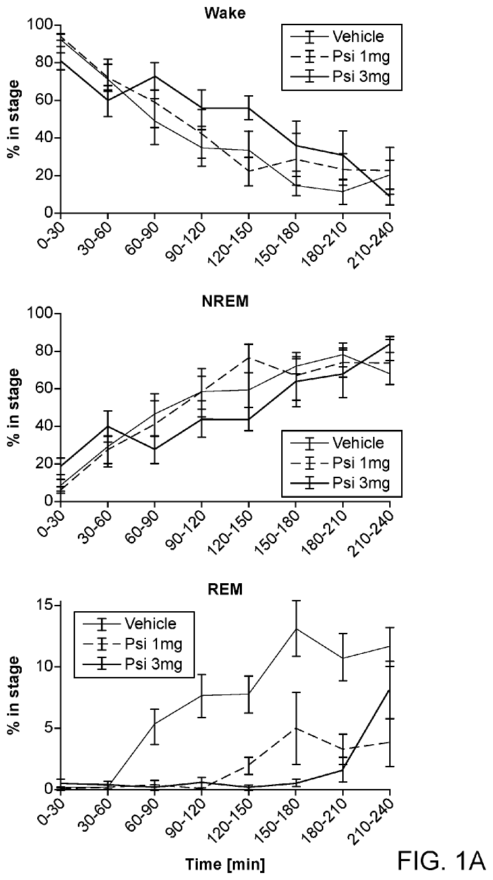

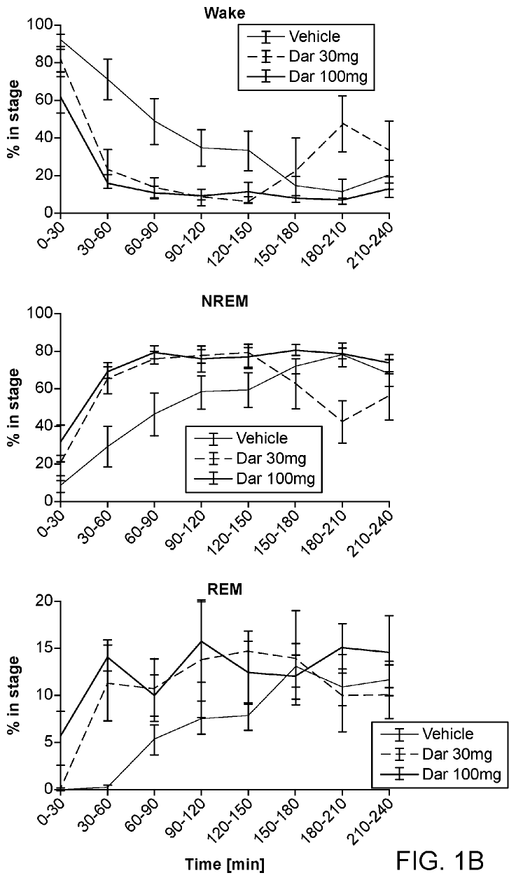

Acute effects were assessed in the first 4 h after dosing by averaging percentage of time spent in each sleep stage (Wake, NREM and REM) across 30-min non-overlapping windows. Psilocybin at 3 mg/kg promoted wakefulness first 60 min after dosing and virtually eliminated REM sleep for 3-4 h after dosing. Similarly, psilocybin at 1 mg/kg reduced REM sleep for 3-4 h after dosing (FIG. 1A). Daridorexant at both 30 and 100 mg/kg acutely reduced wakefulness for the first 2 h after dosing while promoting sleep, especially NREM sleep (FIG. 1B). The combination of daridorexant (30 mg/kg or 100 mg/kg) and psilocybin (1 mg/kg) promoted wakefulness 1-2 h after dosing, while reducing NREM and REM sleep (FIG. 1C), suggesting the ability of psilocybin to override daridorexant-driven wakefulness reduction and sleep promotion.

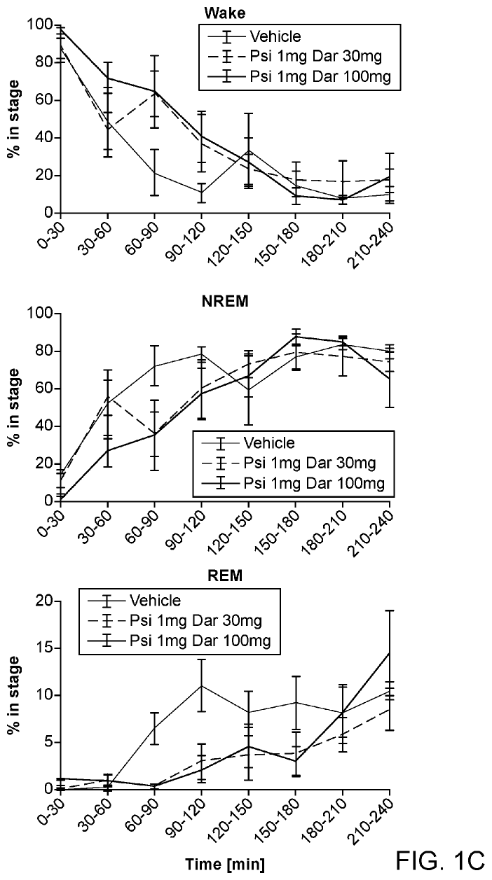

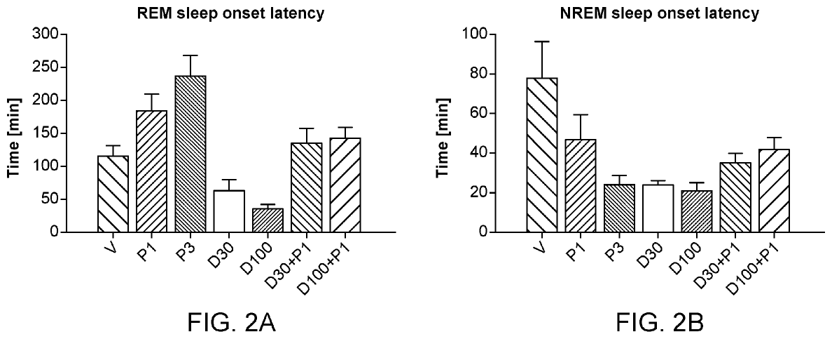

Psilocybin induced a dose-dependent increase in REM onset latency, and a dose-dependent decrease in NREM onset latency (FIG. 2A, B). Daridorexant induced a dose-dependent decrease in REM onset latency and a reduction of NREM onset latency (FIG. 2A, B). Daridorexant and psilocybin combinations exhibited REM sleep onset latency and NREM sleep onset latency comparable to psilocybin alone, suggesting that psilocybin can overcome the effect of daridorexant on sleep onset latency.

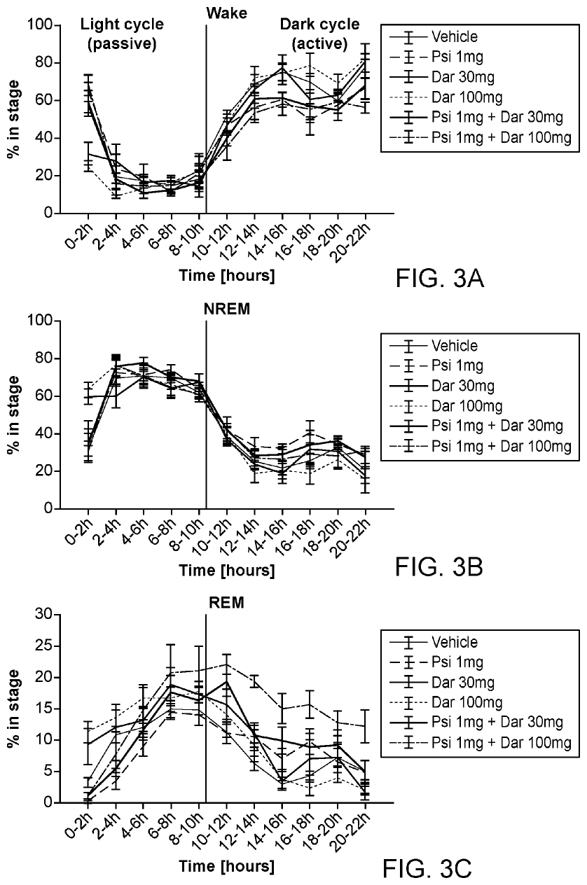

Daridorexant alone caused wakefulness reduction (FIG. 3A) together with promotion of NREM and REM sleep (FIGS. 3B, C) during the first 2 h after dosing. In contrast, psilocybin and combinations of psilocybin and daridorexant had an opposite effect on REM sleep, which was reduced for up to 4 h after dosing, with psilocybin alone having the largest attenuating effect on REM sleep in this time. Surprisingly, in the active period (lights off) starting 10 h and 30 min after dosing, psilocybin and combinations of daridorexant and psilocybin induced a reduction in wakefulness (FIG. 3A) and increases in both NREM and REM sleep (FIG. 3B, C). The effect on REM sleep was most dramatic with the combination of daridorexant 100 mg/kg and psilocybin 1 mg/kg, which induced an increase in REM sleep much greater than either treatment alone (FIG. 3C).

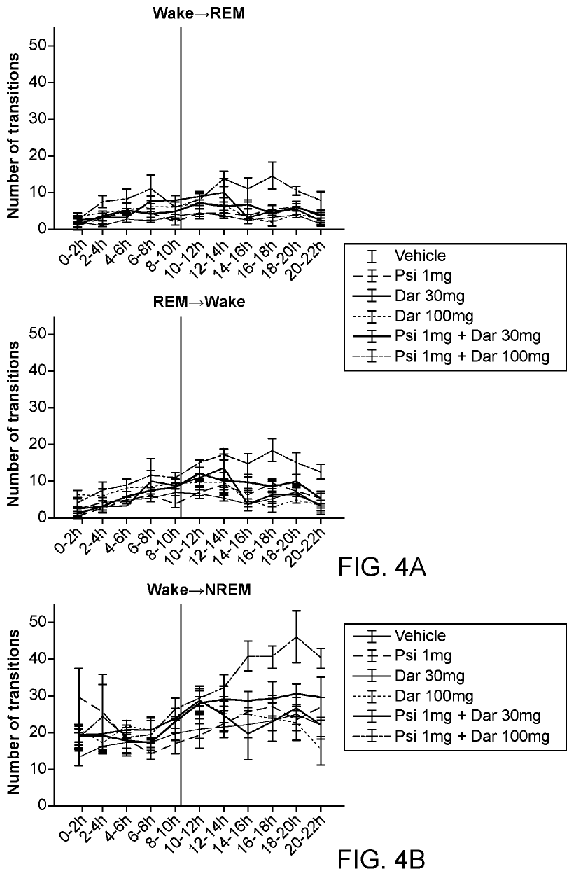

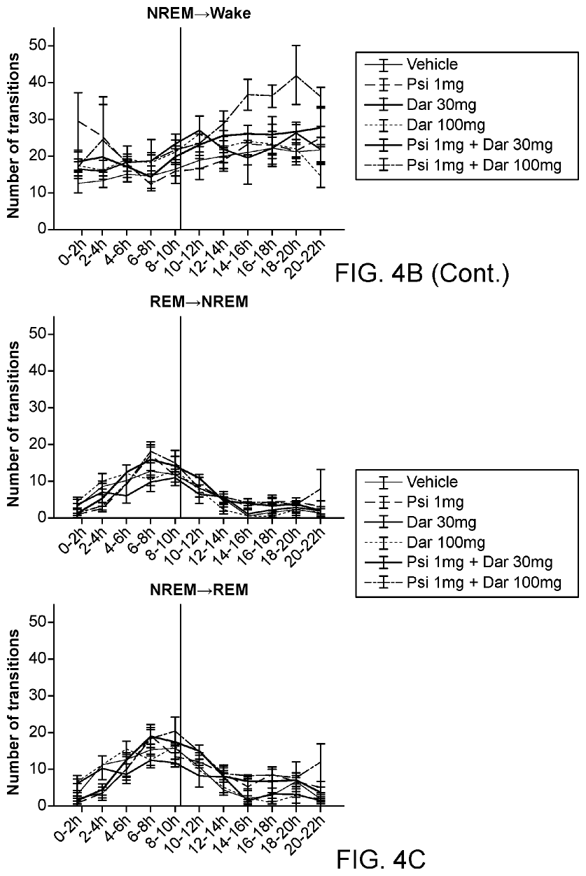

Transitions between wakefulness and REM sleep and REM and NREM sleep were approximately 2-4× rarer than transitions between wakefulness and NREM sleep (FIG. 4). Psilocybin alone induced an increased number of transitions between wakefulness and NREM sleep and a decreased number of transitions between REM and NREM sleep in the first 2 h after dosing. Surprisingly, combinations of daridorexant and psilocybin exhibited increased transitions between wakefulness and REM and between wakefulness and NREM, with the most dramatic increases observed with the combination of daridorexant 100 mg/kg and 1 mg/kg psilocybin, especially throughout the active period (dark cycle).

### Example 3: Head Twitch Response of a 5-HT2A Receptor Agonist in Combination with a Dual Orexin Receptor Antagonist

### Background

Administration of 5-HT2A receptor agonists in mice induces a robust head twitch response (HTR). The number of head twitches observed in mice is recognized as an indicator of the degree of psychedelic effects (Halberstadt et al 2020). To determine whether the HTR induced by 5-HT2A receptor agonists is modulated by dual orexin receptor antagonists, a study was performed comparing the HTR induced by a 5-HT2A receptor agonist, 4-hydroxy-N-methyl-N-ethyltryptamine (4-HO-MET), after pre-treatment with either vehicle or the dual orexin receptor antagonist daridorexant.

### Methods

Male C57BL/6 mice (˜6-8 Week; Hylasco, Hyderabad, India) were pair-housed under standard conditions on a 12-h light cycle with free access to food and water. On the test day, mice were randomly assigned to one of 6 treatment groups (see Table 1 below). Mice were dosed orally with 0.5% methylcellulose or daridorexant at t=−30 min followed by a subcutaneous (s.c.) injection of 4-HO-MET (0.32, 1, or 3.2 mg/kg) at t=0 min. Mice were then immediately placed in a chamber and the number of head twitches in a 20 min period post 4-HO-MET injection (t=0-20 min) were counted manually by a blinded, trained observer.

TABLE 1
Treatment groups for HTR experiment.

| | | Dose (mg/kg) | | Dose | Animals |
|---|---|---|---|---|---|
| Group | Test | 4- | | Volume | per |
| No | Compound ID | HO-MET | Daridorexant | (mL/kg) | Group |
| 1 | 0.5% | 0.32 | — | 10 | 6 |
| 2 | methylcellulose + | 1 | — | 10 | 6 |
| 3 | 4-HO-MET | 3.2 | — | 10 | 6 |
| 4 | Daridorexant + | 0.32 | 100 | 10 | 6 |
| 5 | 4-HO-MET | 1 | 100 | 10 | 6 |
| 6 | | 3.2 | 100 | 10 | 6 |

### Results

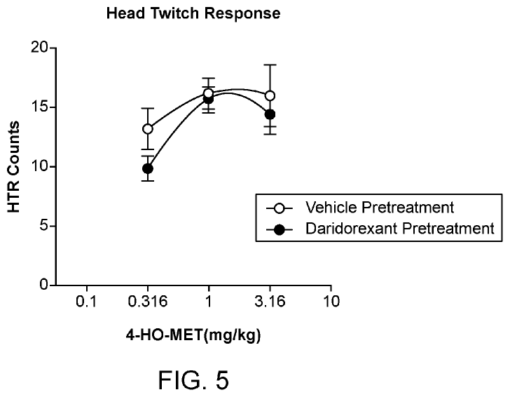

In mice pretreated with vehicle or daridorexant (100 mg/kg), 4-HO-MET (0.32-3.2 mg/kg, s.c.) produced HTR in a dose-dependent manner (FIG. 5). The effects of 4-HO-MET were not significantly affected by pre-treatment with daridorexant.

### CONCLUSION

These results indicate that the HTR of 5-HT2A receptor agonists is not modified by co-administration with a dual orexin receptor antagonist.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1-137. (canceled)

138. A method of treating a neuropsychiatric disorder in a patient in need thereof, comprising administering to the patient a therapeutically effective amount of the combination of an orexin receptor antagonist and either a serotonin receptor agonist or an NMDA receptor antagonist.

139. The method of claim 138, wherein the orexin receptor antagonist is a dual orexin receptor antagonist.

140. The method of claim 138, wherein the orexin receptor antagonist is selected from the group consisting of suvorexant, lemborexant, seltorexant, daridorexant, filorexant, almorexant, and TS-142.

141. The method of claim 140, wherein the orexin receptor antagonist is suvorexant administered in a daily dose between 5 mg to 40 mg, lemborexant administered in a daily dose between 1 mg to 25 mg, seltorexant administered in a daily dose between 1 mg to 45 mg, daridorexant administered in a daily dose between 5 mg to 60 mg, almorexant administered in a daily dose between 50 mg to 500 mg, or TS-142 administered in a daily dose between 5 mg to 30 mg.

142. The method of claim 140, wherein the orexin receptor antagonist is suvorexant administered in a daily dose ranging from about 5 mg to about 30 mg, lemborexant administered in a daily dose ranging from about 5 mg to about 20 mg, seltorexant administered in a daily dose ranging from about 5 mg to about 40 mg, daridorexant administered in a daily dose ranging from about 25 mg to about 50 mg, almorexant administered in a daily dose ranging from about 100 mg to about 400 mg, or TS-142 administered in a daily dose ranging from about 10 mg to about 30 mg.

143. The method of claim 1, wherein the serotonin receptor agonist activates the 5-HT2A receptor.

144. The method of claim 143, wherein the 5-HT2A receptor agonist is 2C-C, 2C-B, 2C-I, 2C-E, 2C-T-2, 2C-T-7, DOM, or mescaline or the 5-HT2A receptor agonist is LSD, AL-LAD, ETH-LAD, or a derivative thereof bearing an acetyl, propionyl, butanoyl, or pentanoyl group on the indole nitrogen.

145. The method of claim 143, wherein the serotonin receptor agonist is selected from a group consisting of a lysergic acid amide, a tryptamine, a phenethylamine, and an amphetamine.

146. The method of claim 145 wherein the serotonin receptor agonist is a lysergic acid amide selected from a group consisting of lysergic acid diethylamide, lysergic acid 2,4-dimethylazetidide (LSZ), 6-ethyl-6-nor-lysergic acid diethylamide (ETH-LAD), 6-propyl-6-nor-Lysergic acid diethylamide (PRO-LAD), 1-Acetyl-N,N-diethyllysergamide (ALD-52), 1-propionyl-lysergic acid diethylamide (1P-LSD), N1-butyryl-lysergic acid diethylamide (1B-LSD), and N1-(cyclopropylmethanoyl)-lysergic acid diethylamide (1 cP-LSD), or is a tryptamine selected from the group consisting of psilocybin, psilocin, N,N-dimethyltryptamine, 5-MeO—N,N-dimethyltryptamine, ibogaine, noribogaine, N-methyl-N-ethyltryptamine (MET), methylisopropyltryptamine (MIPT), diethyltryptamine (DET), diisopropyltryptamine (DIPT), dipropyltryptamine (DPT), ethylpropyltryptamine (EPT), 5-methoxy-methylisopropyltryptamine (5-MeO-MIPT), 5-methoxy-diisopropyltryptamine (5-MeO-DIPT), 5-methoxy-N-methyl-N-cthyltryptamine (5-MeO-MET), 5-methoxy-diethyltryptamine (5-MeO-DET), 4-hydroxy-N-methyl-N-cthyltryptamine (4-HO-MET), 4-hydroxy-methylisopropyltryptamine (4-HO-MIPT), 4-hydroxy-diisopropyltryptamine (4-HO-DIPT), 4-hydroxy-diethyltryptamine (4-HO-DET), 4-hydroxy-dipropyltryptamine (4-HO-DPT), 4-hydroxy-ethylpropyltryptamine (4-HO-EPT), 4-acetoxy-N-methyl-N-ethyltryptamine (4-AcO-MET), 4-acetoxy-methylisopropyltryptamine (4-AcO-MIPT), 4-acetoxy-diisopropyltryptamine (4-AcO-DIPT), 4-acetoxy-diethyltryptamine (4-AcO-DET), 4-acetoxy-dipropyltryptamine (4-AcO-DPT), 4-acetoxy-ethylpropyltryptamine (4-AcO-EPT), 4-acetoxy-dimethyltryptamine (4-AcO-DMT), alpha-methyltryptamine, and alpha-ethyltryptamine, or is a phenethylamine selected from the group consisting of mescaline, escaline, proscaline, methallylescaline, 4-bromo-2,5-dimethoxylpenethylamine (2C-B), 2,5-dimethoxy-4-methylphenethylamine (2C-D), 2-(4-Ethyl-2,5-dimethoxyphenyl)ethanamine (2C-E), 2-(2,5-Dimethoxy-4-propylphenyl)ethan-1-amine (2C-P), 2-[4-(Ethylsulfanyl)-2,5-dimethoxyphenyl]ethan-1-amine (2C-T-2), and 2-[2,5-Dimethoxy-4-(propylsulfanyl)phenyl]ethan-1-amine (2C-T-7) or is an amphetamine selected from the group consisting of 2,5-dimethoxy-4-methylamphetamine (DOM), 2,5-dimethoxy-4-bromoamphetamine (DOB), 2,5-dimethoxy-4-chloroamphetamine (DOC,) 2,5-dimethoxy-4-iodoamphetamine (DOI), 2,5-dimethoxy-4-ethylamphetamine (DOET), and 2,5-Dimethoxy-4-propylamphetamine (DOPR).

147. The method of claim 143, wherein the serotonin receptor agonist is selected from the group consisting of lysergic acid diethylamide, psilocybin, psilocin, 4-acetoxy-dimethyltryptamine (4-AcO-DMT), N,N-dimethyltryptamine, 5-MeO—N,N-dimethyltryptamine, mescaline, 4-bromo-2,5-dimethoxylpenethylamine (2C-B), and 2-(4-Ethyl-2,5-dimethoxyphenyl)ethanamine (2C-E).

148. The method of claim 146, wherein the tryptamine is a 4-hydroxytryptamine or an ester thereof.

149. The method of claim 148, wherein the 4-hydroxytryptamine is psilocin, psilocybin, 4-ACO-DMT, 4-HO-MET, 4-HO-MIPT, 4-HO-DIPT, 4-ACO-MET, 4-ACO-MIPT, or 4-AcO-DIPT.

150. The method of claim 143, wherein the serotonin receptor agonist is 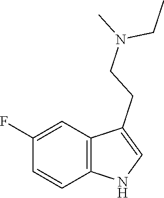 [SMILES: CCN(C)CCc1c[nH]c2ccc(F)cc12]
or a pharmaceutically acceptable salt thereof or is 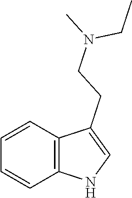
or a pharmaceutically acceptable salt thereof.

151. The method of claim 138, wherein the NMDA receptor antagonist is selected from the group consisting of ketamine, (R)-ketamine, (S)-ketamine and a compound selected from the group consisting of 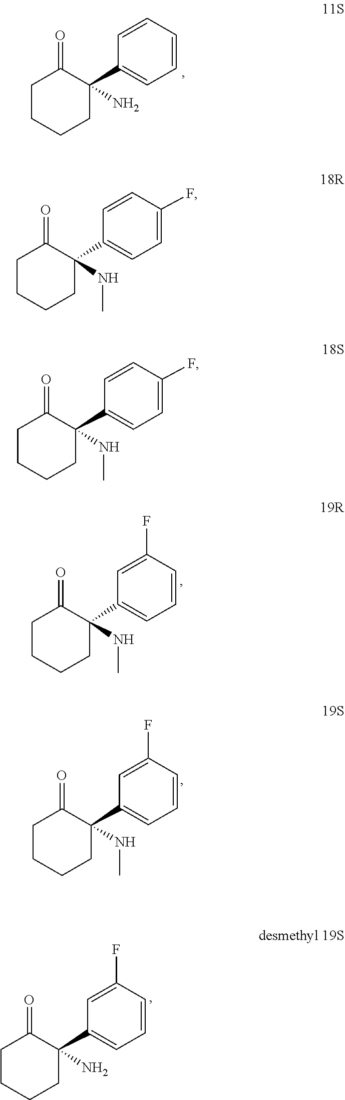 [SMILES: N[C@]1(c2ccccc2)CCCCC1=O ; CN[C@@]1(c2ccc(F)cc2)CCCCC1=O ; CN[C@]1(c2ccc(F)cc2)CCCCC1=O ; CN[C@@]1(c2cccc(F)c2)CCCCC1=O ; CN[C@]1(c2cccc(F)c2)CCCCC1=O ; N[C@]1(c2cccc(F)c2)CCCCC1=O] 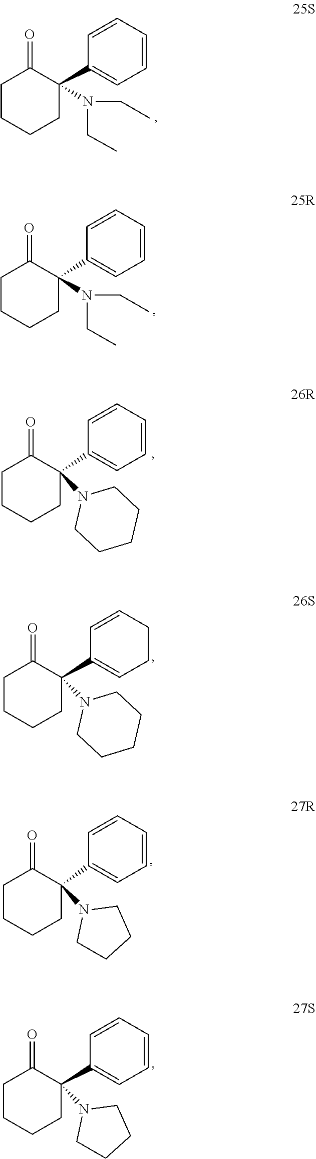 [SMILES: CCN(CC)[C@]1(c2ccccc2)CCCCC1=O ; CCN(CC)[C@@]1(c2ccccc2)CCCCC1=O ; O=C1CCCC[C@]1(c1ccccc1)N1CCCCC1 ; O=C1CCCC[C@@]1(C1=CCCC=C1)N1CCCCC1 ; O=C1CCCC[C@]1(c1ccccc1)N1CCCC1 ; O=C1CCCC[C@@]1(c1ccccc1)N1CCCC1] 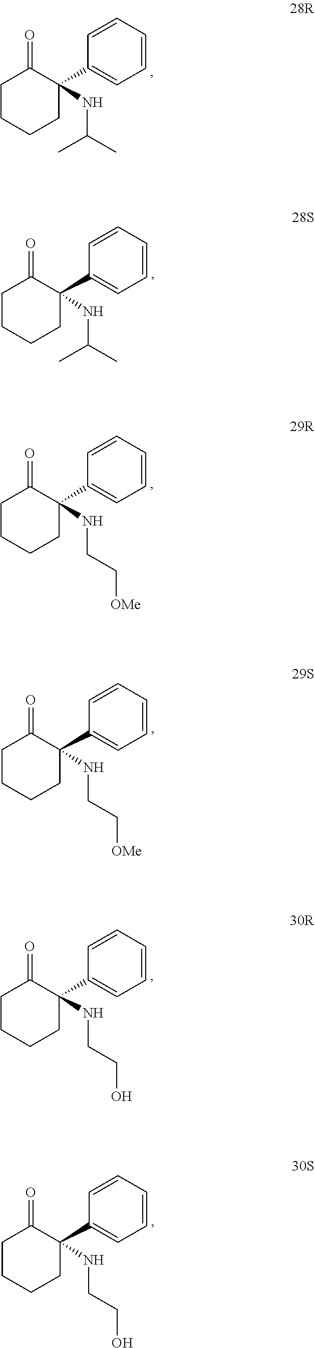 [SMILES: CC(C)N[C@@]1(c2ccccc2)CCCCC1=O ; CC(C)N[C@]1(c2ccccc2)CCCCC1=O ; COCCN[C@@]1(c2ccccc2)CCCCC1=O ; COCCN[C@]1(c2ccccc2)CCCCC1=O ; O=C1CCCC[C@@]1(NCCO)c1ccccc1 ; O=C1CCCC[C@]1(NCCO)c1ccccc1] 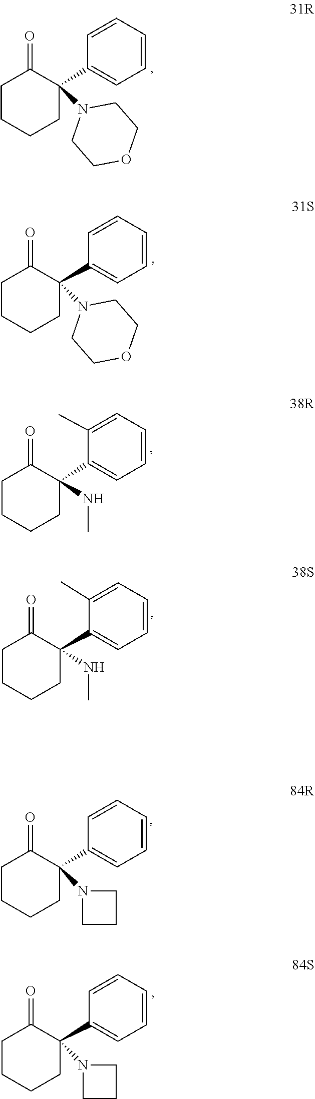 [SMILES: O=C1CCCC[C@]1(c1ccccc1)N1CCOCC1 ; O=C1CCCC[C@@]1(c1ccccc1)N1CCOCC1 ; CN[C@@]1(c2ccccc2C)CCCCC1=O ; CN[C@]1(c2ccccc2C)CCCCC1=O ; O=C1CCCC[C@]1(c1ccccc1)N1CCC1 ; O=C1CCCC[C@@]1(c1ccccc1)N1CCC1] 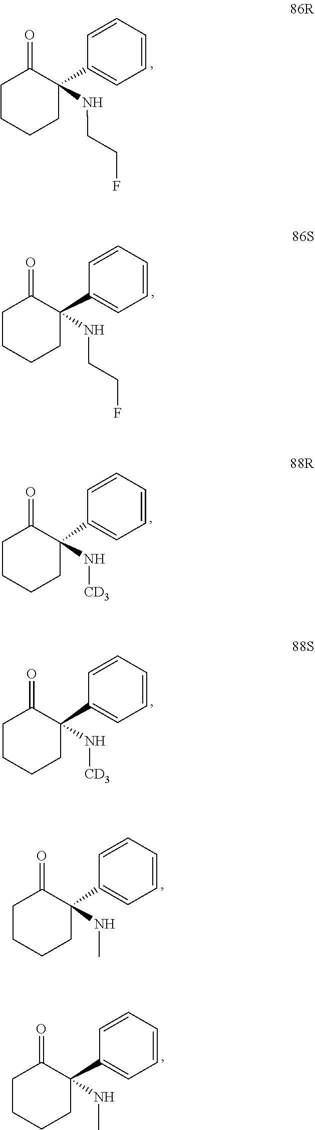 [SMILES: O=C1CCCC[C@@]1(NCCF)c1ccccc1 ; O=C1CCCC[C@]1(NCCF)c1ccccc1 ; [2H]C([2H])([2H])N[C@@]1(c2ccccc2)CCCCC1=O ; [2H]C([2H])([2H])N[C@]1(c2ccccc2)CCCCC1=O ; CN[C@@]1(c2ccccc2)CCCCC1=O ; CN[C@]1(c2ccccc2)CCCCC1=O] 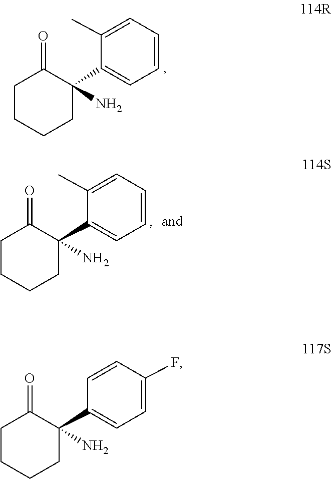
or a pharmaceutically acceptable salt thereof.

152. The method of claim 143, wherein the serotonin receptor agonist is psilocybin.

153. The method of claim 152, wherein the psilocybin is administered orally at a dose of 1-40 mg or is administered intranasally at a dose of 1-40 mg.

154. The method of claim 143, wherein the serotonin receptor agonist is administered in a dose selected to induce a subperceptual or threshold psychoactive effect in the patient.

155. The method of claim 151, wherein the NMDA receptor antagonist is administered in a dose selected to induce a dissociative effect in the patient.

156. The method of claim 138, wherein the orexin receptor antagonist and the serotonin receptor agonist or NMDA receptor antagonist are administered simultaneously or are administered as a fixed-dose combination.

157. The method of claim 156, wherein the orexin receptor antagonist and the serotonin receptor agonist or NMDA receptor antagonist are administered orally, intranasally, or by vaporization.

158. The method of claim 152, wherein the psilocybin is administered as an oral dosage form, wherein, the oral dosage form comprises:
crystalline psilocybin in the form Polymorph A characterized by peaks in an XRPD diffractogram at 11.5, 12.0, 14.5, 17.5, and 19.7°2θ±0.1°2θ, wherein the crystalline psilocybin has a chemical purity of greater than 97% by HPLC, and silicified microcrystalline cellulose.

159. The method of claim 158, wherein the oral dosage form comprises 1 mg to 40 mg of crystalline psilocybin in the form Polymorph A.

160. The method of claim 138, wherein the effective amount of a serotonin receptor agonist and/or an NMDA receptor antagonist is administered within about 0-120 minutes before bedtime

161. The method of claim 138, wherein the method further comprises administering a behavioral approach therapy to the patient.

162. The method of claim 161, wherein the behavioral approach therapy is selected from the group consisting of dream incubation, preparation critical learning, and prior day cognitive task performance.

163. The method of claim 138, wherein the neuropsychiatric disorder is a mood disorder.

164. The method of claim 163, wherein the mood disorder is depression.

165. The method of claim 164, wherein the depression is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, prolonged or pathological grief, and depressive disorder due to another medical condition.

166. The method of claim 164, wherein the depression is depression with ruminations, depression with anhedonia, depression with anxiety, or depression with sleep disturbance.

167. The method of claim 163, wherein the mood disorder is bipolar disorder, a substance-related disorder, a substance-use disorder, an anxiety disorder, obsessive-compulsive and related disorders, trauma- and stressor-related disorders, feeding and eating disorders, borderline personality disorder, attention-deficit/hyperactivity disorder, and autism spectrum disorder.

168. The method of claim 138, wherein the neuropsychiatric disorder is a neurocognitive disorder.

169. The method of claim 168, wherein the neurocognitive disorder is selected from the group consisting of neurocognitive disorder, mild neurocognitive disorder, major or mild neurocognitive disorder due to Alzheimer's disease, major or mild frontotemporal neurocognitive disorder, major or mild neurocognitive disorder with Lewy bodies, major or mild vascular neurocognitive disorder, major or mild neurocognitive disorder due to traumatic brain injury, substance/medication-induced major or mild neurocognitive disorder, major or mild neurocognitive disorder due to HIV infection, major or mild neurocognitive disorder due to prion disease, major or mild neurocognitive disorder due to Parkinson's disease, major or mild neurocognitive disorder due to Huntington's disease, major or mild neurocognitive disorder due to another medical condition, and major or mild neurocognitive disorder due to multiple etiologies.

170. The method of claim 138, wherein the method provides improvement in at least one symptom selected from the group consisting of sadness or lethargy or lassitude, depressed mood, inability to feel, anxious worried feelings, fears, feeling tense, feeling restlessness, diminished interest in all or nearly all activities, difficulty initiating activities, significant increased or decreased appetite leading to weight gain or weight loss, insomnia, sleep disturbance, irritability, fatigue, feelings of worthlessness or low self-esteem, strongly held negative beliefs or pessimistic thoughts about self, others or world, feelings of helplessness, inability to concentrate or distractibility, suicidality, feelings of guilt, memory complaints, difficulty experiencing positive feelings, feeling cut off or distant from people, hypervigilance, risk taking behavior, avoidance of thoughts about a stressful or traumatic event, pains and aches, ruminations and obsessive thoughts, compulsive behaviors, talking to people you don't know well or strangers, being center of attention, disturbing intrusive thoughts, can't get through week without drug use, guilty about drug use, problems with friends or family due to drug use, and withdrawal symptoms due to substance use.

171. A method of improving sleep in a patient in need thereof, comprising administering to the patient a therapeutically effective amount of a combination of an orexin receptor antagonist and a serotonin receptor agonist or an NMDA receptor antagonist.

172. The method of claim 171, wherein the sleep is slow wave sleep or REM sleep.

173. A method of improving memory in a patient in need thereof, comprising administering to the patient a therapeutically effective amount of a combination of an orexin receptor antagonist and a serotonin receptor agonist or an NMDA receptor antagonist.

174. A method of improving learning in a patient in need thereof, comprising administering to the patient a therapeutically effective amount of a combination comprising an orexin receptor antagonist and a serotonin receptor agonist or an NMDA receptor antagonist.

175. The method of claim 167, wherein the substance use disorder is an addiction to a substance with a symptom of withdrawal due to substance use.

176. The method of claim 175, wherein the substance is selected from the group consisting of stimulants selected from the group consisting of cocaine, amphetamines, methamphetamines, methylphenidate, and related stimulants; opioids selected from the group consisting of heroin, fentanyl, codeine, hydrocodone, and related opioid drugs; nicotine; alcohol, prescription medications selected from the group consisting of sedative-hypnotic drugs, medications prescribed for pain management selected from oxycodone, hydrocodone and other non-opioid pain medicines; naturally-occurring plant-derived drugs selected from the group selected from marijuana, tobacco, and the addictive agents therein; and synthetic drugs.

177. The method of claim 167, wherein the substance use disorder is nicotine addiction and/or smoking cessation.

178. The method of claim 138, wherein the orexin receptor antagonist is suvorexant administered in a daily dose between 5 mg to 40 mg, lemborexant administered in a daily dose between 1 mg to 25 mg, seltorexant administered in a daily dose between 1 mg to 45 mg, daridorexant administered in a daily dose between 5 mg to 60 mg, almorexant administered in a daily dose between 50 mg to 500 mg, or TS-142 administered in a daily dose between 5 mg to 30 mg. and the serotonin receptor agonist is psilocybin, wherein the psilocybin is administered orally at a dose of 1-40 mg or is administered intranasally at a dose of 1-40 mg.

179. The method of claim 178 wherein the psilocybin is crystalline psilocybin administered orally in the form of Polymorph A.
